(19) United States
(12) Patent Application Publication
(10) Pub. No.: US 2004/0024005 A1
(43) Pub. Date: Feb. 5, 2004

(54) Methods and compositions for reducing the development of drug tolerance and/or physical dependence

(75) Inventors: Jennifer Whistler, El Cerrito, CA (US); Mark von Zastrow, San Carlos, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/350,270

(22) Filed: Jan. 22, 2003

Related U.S. Application Data
(60) Provisional application No. 60/351,466, filed on Jan. 23, 2002.
(60) Provisional application No. 60/351,442, filed on Jan. 23, 2002.

(51) Int. Cl. A61K 31/485

(52) U.S. Cl. 514/282

(57) ABSTRACT

The present invention provides methods for reducing, preventing or delaying the development of tolerance to certain drugs that target G-protein coupled receptors (GPCR). The methods are generally carried out by co-administering with the drug an agonist for the drug-target GPCR that promotes the endocytosis of the targetted receptor. The methods are particularly useful for drugs that target the opioid receptors, for example morphine. The present invention also provides compositions comprising a drug and an agonist that are advantageous in preventing the development of tolerance to the drug that can develop when the drug is administered alone.

### CROSS-REFERENCE TO RELATED APPLICATIONS

This application claims the benefit of U.S. Provisional Application No. 60/351,442 and 60/351,466, both filed Jan. 23, 2002.

### STATEMENT AS TO RIGHTS TO INVENTIONS MADE UNDER FEDERALLY SPONSORED RESEARCH AND DEVELOPMENT

This invention was made with government support under NIH grant DA10711 and NRSA grant DA05844. As such, the government may have certain rights in this invention.

### FIELD OF THE INVENTION

The present invention relates to generally to the area of methods and compositions for reducing the development of drug tolerance. In particular, the invention relates to methods and compositions for reducing the development of tolerance to drugs that target G protein-coupled receptors.

### BACKGROUND OF THE INVENTION

Opioid receptors belong to the large superfamily of G protein-coupled receptors (GPCRs). GPCRs, which are found in abundance in organisms as diverse as vertebrates, nematodes, plants, yeast, and slime mold, as well as in protozoa and the earliest diploblastic metazoa, are of fundamental physiological importance because they mediate the physiological actions of the majority of known neurotransmitters and hormones. A useful review of the properties of GPCRs can be found in Bockaert and Pin, EMBO J. 1999, Vol. 18, pp. 1723-1729. GPCRs share a common structural feature of a central core domain constituted of seven transmembrane helices connected by three intracellular and three extracellular loops. They have been classified into five or six families based on sequence similarities and each family is further divided into a number of subfamilies (see, Bockaert and Pin, supra). GPCR family 1 contains the largest number of known receptors including the rhodopsin, adenosine, adrenergic, serotonin and opioid receptors.

Opioid receptors are particularly intriguing members of the GPCR receptor class because they are activated both by endogenously produced opioid peptides and by exogenously administered opiate drugs (Hughes et al. (1983) British Medical Bulletin 39:1-3), which are the most effective analgesics known as well as highly addictive drugs of abuse. While opiates such as morphine remain the analgesic of choice in many cases, a major limitation to their long-term use is the development of tolerance, which is a profound decrease in analgesic effect observed in most patients during prolonged administration of opiate drug. In addition, long-term use of opiates causes physical dependence in some patients, which is a requirement for continued administration of increasing doses of drug to prevent the development of symptoms of opiate withdrawal. Despite considerable progress, the molecular and cellular mechanisms mediating the development of tolerance and dependence to morphine remain controversial (Nestler (1996) Neuron 16:897-900; Nestler (2001) Nat Rev Neurosci 2:119-128; Williams et al. (2001) Physiol Rev 81:299-343).

Studies using knockout mice confirm that opiate analgesia and dependence are mediated by mu opioid receptors (MORs) (Matthes et al. (1996) Nature 383:819-823). Following activation by either alkaloid or peptide agonist, opioid receptors are regulated by multiple mechanisms, including a well-characterized and highly conserved process involving receptor phosphorylation by G protein coupled receptor kinase (GRK) and subsequent arrestin recruitment (reviewed in Ferguson (2001) Pharmacol Rev 53:1-24). These processes can contribute directly to GPCR desensitization by facilitating the uncoupling of receptor from G protein. Following this desensitization, receptors are often endocytosed into an intracellular compartment from which they can be recycled to the membrane leading to receptor resensitization or targeted for degradation leading to receptor downregulation (Lefkowitz et al. (1998) Advances in Pharmacology 42:416-420). Hence these processes can contribute directly to tolerance by decreasing the number of functional cell surface receptors. Consequently, one current view is that opioid receptor desensitization and endocytosis contribute directly to physiological tolerance by reducing the number of functional receptors present.

Another view of the development of tolerance, suggested by earlier work of some of the present inventors (Whistler et al. (1999) Neuron 23:737-746), is that in some cases, endocytosis plays a protective role in reducing the development of tolerance. This hypothesis is based on the observations that morphine-activated MORs elude GRK phosphorylation and subsequent arrestin binding and desensitization (Blake et al. (1997) J Biol Chem 272:782-790; Whistler, et al. (1998) Proc Natl Acad Sci USA 95:9914-9919; Zhang et al. (1998) Proc Natl Acad Sci USA 95:7157-62). Additionally, morphine fails to promote endocytosis of the wild type MOR in cultured cells (Arden et al. (1995) J Neurochem 65:1636-1645; Keith et al. (1996) J Bio Chem 271:19021-19024) and native neurons (Keith et al. (1998) Molecular Pharmacology 53:377-384; Sternini et al. (1996) Proc Natl Acad Sci USA 93:9241-9246), whereas endogenous peptide ligands such as endorphins and several opiate drugs such as methadone readily drive receptor endocytosis (Trapaidze et al. (2000) Brain Res Mol Brain Res 76:220-8). Furthermore, numerous studies have demonstrated no substantial downregulation in the number of MORs even in profoundly morphine tolerant animals (for example (De Vries et al. (1993) Life Sci 52:1685-1693); Simantov et al. (1984) Neuropeptides 5:197-200) and reviewed in (Williams et al. (2001) Physiol Rev 81:299-343). Recent work by one of the present inventors demonstrated that MOR mutations that facilitate endocytosis reduce the development of cellular tolerance and cAMP superactivation, a cellular hallmark of withdrawal, in a cell culture model (Finn, et al. (2001) Neuron 32:829-839).

Recently, several groups have reported dimerization of a number of GPCRs including the dopamine and serotonin receptors (Lee S. P. et al. (2000) Neuropsychopharmacology 23:S32-40), the beta2-adrenergic receptor (Angers S. et al. (2000) Proc Natl Acad Sci USA 97:3684-3689), and the opioid receptors (Jordan B. A. et al. (1999) Nature 399:697-700). And in fact, heterodimerization of opioid receptors has been shown to alter opiate ligand properties (Jordan B. A. et al. (1999) Nature 399:697-700) and affect receptor trafficking (Jordan B. A. et al. (2001) Proc Natl Acad Sci USA 98:343-348). It has been suggested that homo- and hetero-dimers of the GPCRs are involved in modulating the function of the receptors and thus are important for the development and screening of new drugs (Salahpour et al. 2000 Trends Endocrinol Metab 11:163; Devi LA 2001 Trends Pharmacol. Sci 22:532).

The importance of morphine as an analgesic has lead to the development of a number of approaches to prevent or reduce the development of tolerance. U.S. Pat. No. 4,575,506 describes the use of thiamphenicol to inhibit the development of tolerance to morphine and other central analgesic agents. U.S. Pat. No. 4,416,871 describes the use of certain dipeptides to inhibit morphine tolerance. U.S. Pat. No. 5,057,519 describes a method for delaying the onset of opiate tolerance by administration of benzamide type 5-HT3 receptor antagonists. U.S. Pat. No. 5,183,807 describes the use of ganglioside GM1 to prevent development of tolerance to morphine. U.S. Pat. No. 5,352,680 describes treating opiate tolerance with certain delta opioid receptor antagonists. U.S. Pat. No. 5,908,832 describes treating opiate addiction by administering certain peptide analogs of neuropeptide FF. U.S. Pat. No. 5,041,446 describes a method of inhibiting development of morphine tolerance by administering dapiprazole. U.S. Pat. No. 5,654,281 describes a method of inhibiting the development of tolerance to addictive substances using an NMDA receptor antagonist. U.S. Pat. Nos. 5,472,943, 5,580,876, 5,767,125 and RE36547 describe methods for enhancing the potency of certain bimodally-acting opioid agonists and attenuating undesirable side effects by administering certain opioid antagonists in combination with the agonists.

However, despite the vast amount of research aimed at preventing morphine tolerance, this remains a serious limitation to the use of morphine as an analgesic. Methods for preventing or delaying the development of tolerance to morphine, as well as tolerance to a number of other GPCR-acting drugs, would be highly desirable.

### SUMMARY OF THE INVENTION

The invention provides a composition useful for reducing, preventing or delaying the development of tolerance to, and/or physical dependence on, particular drugs that target G-protein coupled receptors (GPCRs). Compositions of the invention are generally pharmaceutical compositions including: a drug that targets a GPCR, wherein the drug does not promote endocytosis and resensitization of the targetted GPCR; an agonist for the GPCR, wherein the agonist promotes the endocytosis of the GPCR and is present in the composition in an amount sufficient to promote endocytosis and resensitization of the targetted GPCR; and a pharmaceutically acceptable carrier. In preferred compositions, the drug activates the GPCR.

In one embodiment, a composition of the invention comprises a drug that includes an analgesic, which is present in the composition in an analgesic amount. In a variation of this embodiment, the composition comprises an agonist that includes an analgesic, which is present in the composition in a sub-analgesic amount. In a preferred embodiment, the drug comprises an opioid drug, the GPCR comprises the mu opioid receptor, and the agonist comprises a mu opioid receptor agonist. For example, the opioid drug can include morphine, and the agonist can include a mu opioid receptor agonist selected from DAMGO, methadone, fentanyl, sufentanil, remi-fentanyl, etonitazene, and etorphine.

The present invention additionally provides a method for reducing, preventing or delaying the development of tolerance to, and/or physical dependence on, particular drugs that target G-protein coupled receptors (GPCRs) by co-administering the drug with an agonist for the receptor. Tolerance and/or physical dependence develops as a consequence of the failure of the drug to promote endocytosis (with subsequent resensitization) of the target receptor. GPCR-targetting drugs useful in the treatment method invention can either activate or block the activity of the targetted receptor.

The agonist to be co-administered is one that promotes endocytosis of the drug receptor target. The agonist is administered in an amount sufficient to promote endocytosis to the GPCR in the subject, whereby the drug targetted GPCR is endocytosed and resensitized. Co-administration of the drug and the agonist can be achieved in a subject by (1) administering the drug to a subject receiving the agonist; (2) administering the agonist to a subject receiving the drug; or (3) administering the drug and the agonist to a subject.

In one embodiment of this treatment method, the drug includes an analgesic, which present in the subject in an analgesic amount. In a variation of this embodiment, the agonist includes an analgesic, which is present in the subject in a sub-analgesic amount. In a preferred embodiment, the drug comprises an opioid drug, the GPCR comprises the mu opioid receptor, and the agonist comprises a mu opioid receptor agonist. For example, the opioid drug can include morphine, and the agonist can include a mu opioid receptor agonist selected from DAMGO, methadone, fentanyl, sufentanil, remi-fentanyl, etonitazene, and etorphine.

Another aspect of the invention is a method of screening for an agent that reduces, prevents or delays the development of tolerance to, and/or physical dependence on, a drug that targets a GPCR. The screening method entails: (1) contacting a test agent with a cell comprising the GPCR; (2) determining whether the test agent promotes the endocytosis of the GPCR; and (3) selecting a test agent that promotes endocytosis of the GPCR as an agent that may reduce, prevent or delay the development of tolerance and/or physical depenedence to the drug. The test agent is preferably contacted with the cell in vitro to facilitate screening. In a preferred embodiment, endocytosis is determined by a ligand binding assay.

In one embodiment of this screening method, the test agent includes an analgesic. In a preferred embodiment, the GPCR comprises the mu opioid receptor, and the test agent comprises a mu opioid receptor agonist.

In a preferred embodiment, the method additionally includes recording any selected test agent in a database of agents that may reduce, prevent or delay the development of tolerance to, and/or physical dependence on, the drug.

Other embodiments of the screening method include combining a test agent selected for promoting endocytosis with a pharmaceutically acceptably carrier and optionally adding a drug that targets the GPCR), wherein the drug does not promote endocytosis of the GPCR.

These and other aspects of the invention will be apparent from the detailed description herein.

### BRIEF DESCRIPTION OF THE DRAWINGS

FIG. 1A. Immunoblots of receptor oligomers. HEK 293 cells were stably transfected with constructs containing both FLAG-MOR and HA-DMOR and treated with morphine (MS), etorphine (ET) or left untreated (NT). Cell were permeabilized and the receptors were immunoprecipitated with anti-HA antibodies (upper panel) or anti-FLAG antibodies (lower panel). Cells expressing only FLAG-MOR (upper panel-left lane) or no receptors (293, lower panel-left lane) were used as controls.

FIGS. 1B and 1C. Fluorescent microscopy of stably transfected HEK293 cells showing the localization of the receptors. Cells stably expressing either FLAG-MOR (F-MOR), or HA D MOR (HA-D MOR) (single stables, 1B), or both receptors (double stables, 1C) were fed antibody to the extracellular epitope tag of the receptor and examined for receptor distribution following morphine treatment (5 μM, 30 minutes). MORs were distributed primarily on the cell surface in cells expressing only MOR whereas D MORs were redistributed to endocytic vesicles. In cells co-expressing both receptors, not only D MORs but also MORs were redistributed to endocytic vesicles, with a significant number of vesicles showing colocalization of both receptors (yellow in right panel).

FIG. 2. Fluorescent microscopy of transfected neurons. Three week old hippocampal cultures were transfected with FLAG-MOR, HA-D MOR or both receptors. Neurons were then examined for receptor distribution following antibody feeding and morphine treatment (5 μM, 30 minutes) by staining with antibodies to the extracellular epitope tag of each receptor type, anti-FLAG for MOR and anti-HA for D MOR). MORs in neurons expressing only this receptor were distributed primarily on the cell surface (upper left panel). D MORs were rapidly redistributed to endocytic vesicles upon morphine activation (upper right panel). In neurons that co-expressed MOR and D MOR, both receptors were redistributed to endocytic vesicles following activation by morphine (lower panels, anti-FLAG on the left, anti-HA on the right).

FIG. 3. Fluorescent microscopic analysis of MOR-transfected HEK293 cells. HEK 293 cells expressing FLAG-MOR were analyzed by antibody staining using an anti-FLAG antibody for receptor distribution following treatment with various agonists. A saturating concentration of DAMGO (5 μM, 30 minutes) promoted robust endocytosis of MOR (upper left panel), whereas morphine at the same dose had little effect on receptor distribution (upper right panel) with the receptor remaining predominantly at the cell surface. A sub-saturating dose of DAMGO (100 nM) caused less endocytosis than that seen with the saturating 5 μM dose (lower left panel). However, this sub-saturating dose of DAMGO (100 nM), when administered concurrently with a saturating dose of morphine (5 μM), facilitated robust endocytosis of the MOR (lower right panel).

FIG. 4. Fluorescent microscopic analysis of HEK293 cells stably transfected with FLAG-MOR and HA-B2AR. Cells stably expressing FLAG-MOR and HA-B2AR were fed antibody to the extracellular epitope tags of the receptors and examined for receptor distribution following various agonist treatments (all 5 μM, 30 minutes). Anti-FLAG antibody signal shown in the left panels, anti-HA antibody signal shown in the center panels, right panels show the merge of the two antibody signals. (A) No agonist; (B) DAMGO; (C) Isoproterenol; (D) DAMGO and Isoproterenol; (E) Morphine and isoproterenol.

FIG. 5. Relative luciferase activity in HEK293 cells transfected with a CRE-luciferase reporter gene and FLAG-MOR. Cells stably expressing MOR and a CRE-luciferase reporter gene were treated chronically (14 hours) with morphine (MS) at 1 μM, DAMGO (DG) at 1 μM, 100 nM, 10 nM or 1 nM, or both drugs (1 μM morphine+10 nM DAMGO) and superactivation of the cAMP pathway was assessed relative to untreated cells (NT). Morphine (1 μM) caused pronounced superactivation. DAMGO also caused superactivation in a dose dependent manner. A dose of DAMGO that caused little superactivation (10 nM) when administered alone, when administered concurrently with the superactivation-inducing dose of morphine (1 μM) reduced the morphine-induced superactivation. P<0.01, two-way ANOVA, Tukey's post test.

FIG. 6. (A) Tail-flick latencies (sec) before (white bars) and 30 min. after (black bars) drug administration via IT catheter were measured. Acute doses of DAMGO (DG) at 0.01 nmoles, and 0.3 nmoles and morphine (MS) at 30 nmoles were used. (B) Immunohistochemical staining of MORs in the lamina II neurons of the spinal cord proximal to the catheter following the behavior testing. MORs were redistributed to endocytic compartments following treatment with 0.3 nmoles of DAMGO (upper left panel). Little endocytosis was observed following treatment with the equi-analgesic dose of morphine (30 nmoles—upper right panel), or with the sub-analgesic dose of DAMGO (0.01 nmoles—lower left panel). However, MORs in rats treated simultaneously with 0.01 nmoles of DAMGO and 30 nmoles of morphine were redistributed to endocytic vesicles (lower right panel). Quantification of vesicles is listed below each image and was achieved by encoding the slides, and having a second party count vesicles from a center section of a Z stack for at least 8 cells per condition from 2 rats per condition.

FIG. 7. (A) Tail-flick latency measurement over time course of morphine tolerance development. Rats were implanted with an IT catheter and a time course of morphine tolerance development was assessed with daily tail flick latency testing before pump implantation (day 0) and for 7 consecutive days. Morphine (MS) was chronically infused at 2, 6, or 18 nmoles/hr. Morphine induced tolerance at all three doses. Equal volume saline was used as control. (B) Rats were implanted with a Y-shaped IT catheter. One arm of the catheter was attached to a mini pump that was prefilled with morphine and implanted subcutaneously. The other arm of the catheter was used for daily injection of DAMGO or saline. 0.01 nmoles of DAMGO/15 μl or the same volume of saline were injected twice per day. Analgesia was assessed by tail flick latency test once per day 30 minutes following the second injection. The results were analyzed by two-way ANOVA followed by Bonferroni post-test (* p<0.05, ** p<0.01, *** p<0.001 vs. saline; $ p<0.05, $$ p<0.01, significantly different from MS 6 nmole+saline group. N=4-6 per group, mean±SEM are shown). There was no significant difference between the MS 6 nmole+DG 0.01 nmole group and the MS 6 nmole+saline group for the first 3 days (p>0.05). (C) Immunohistochemical staining of MORs in the lamina II neurons of the spinal cord from the rats of (B). MORs were primarily localized to the plasma membrane of neurons of rats treated with saline, morphine or the low dose of DAMGO. However, pronounced MOR endocytosis was observed following co-administration of morphine with the low dose of DAMGO. Quantification was achieved by encoding the slides, and having a second party count vesicles from a center section of a Z stack for at least 8 cells from 2 rats per condition.

FIG. 8. Tail-flick latency measurement over the time course of morphine tolerance development. Rats were implanted with an introcerebroventricular (i.c.v.) cannula and a time course of morphine tolerance development was assessed with daily tail flick latency testing before pump implantation (day 0) and for 7 consecutive days. Equal volume saline was used as a control. Morphine (MS) was chronically infused at 25 or 75 nmoles/hr. Morphine induced tolerance at both doses.

FIG. 9. Measurement of symptoms of withdrawal after 7 days of i.c.v. morphine. After mini-pump implantation, morphine or saline was infused chronically for 7 consecutive days. Morphine (MS) was infused at 25 or 75 nmoles/hr. Equal volume saline was used as a control. On day 7, rats were injected intraperitoneally (i.p.) with 3 mg/kg naloxone and placed, individually, in Plexiglass cylinders. The rats were monitored for jumping, shaking, and chewing, and the number of occurrences of each type of behavior over a 20-min period was recorded immediately following the naloxone injection. In addition, the rats were weighed before naloxone injection and after the 20 mins of monitoring indicated above, and percentage weight loss was calculated. Morphine treatment produced symptoms of withdrawal indicating that physical dependence had developed at both doses.

FIG. 10. Tail-flick latency produced by morphine, as compared to DAMGO, before and after induction of tolerance. Rats were implanted with i.c.v. cannulae (but not with mini-pumps). Morphine (MS, 50 nmoles) or DAMGO (DG, 1 nmole) was given directly via cannula, twice a day: morning and afternoon, in 5 μl volume for 5 days. Rats were tested for analgesia using the tail-flick latency test twice during the study period. The tests were conducted 30 min after the morning dose on days 1 and 5. Maximum Possible Effect (MPE) was calculated using the following equation: (Post-drug latency—baseline latency)/(cut-off latency—baseline latency)×100%. “Cut-off latency” refers to the time at which the laser was automatically shut off to prevent tissue damage to animals fully analgesic. The percent MPE is shown for tail flick latency tests conducted after the initial morphine (MS 50 nmol) and DAMGO (DAMGO 1.0 nmol) treatments and after 5 days of treatment with morphine (ms 50 after ms 50) and DAMGO (DG 1.0 after DG 1.0). These data indicate that DAMGO produces less tolerance than morphine.

FIG. 11. Symptoms of withdrawal produced by morphine, as compared to DAMGO, after 5-day treatment. Rats were treated i.c.v. with morphine (MS, 50 nmoles) and DAMGO (1.0 nmole) twice daily for 5 days as described for FIG. 10. On day 5, 30 mins following the second drug administration, rats were injected intraperitoneally with 3 mg/kg naloxone and placed, individually, in Plexiglass cylinders. The rats were monitored for jumping, shaking, and chewing, and the number of occurrences of each type of behavior over a 20-min period was recorded immediately following the naloxone injection. In addition, the rats were weighed before naloxone injection and following the 20-min observation period indicated above. The results indicate that, by four indicators of withdrawal, DAMGO produces less withdrawal, indicating less physical dependence, than morphine.

FIG. 12. Rats were treated chronically i.c.v. with 25 or 75 nmoles/hr of morphine or saline administered from a mini-pump for 7 consecutive days, as described in Examples 9.A. and 9.B. to induce tolerance After the behavioral study described in Example 9.B., and the brains were quickly removed and frozen by immersion in isopentane on dry ice and then stored at −80° C. Brain sections, 16 μm thick, were cut on a cryostat at −18° C., thaw-mounted onto slides, and stored desiccated at −800° C. To quantitate the number of MOR receptors in these sections, a [³H]-DAMGO binding assay was carried out using slides containing sections from the midbrain, forebrain, and brain stem as described in Example 9.E. Brain areas in autoradiograms were quantitated using NIH Image software, and optical densities were converted into fmol/mg tissue according to commercial standards exposed adjacent to the brain sections.

FIG. 12 is a histogram showing the results of this study for different brain regions: the striatum, the nucleus accumbens (Nac), the hippocampus, the thalamus, the amygdala, and the brain stem (PAG). Results are shown for rats treated for 7 days with saline (naïve) or 25 or 75 nmoles/hr morphine (MS 25 nmol and MS 75 nmol, respectively), administered via mini-pump. Chronic morphine treatment sufficient to induce tolerance does not result in a reduction in receptor number. In fact, chronic morphine treatment was correlated with a significant increase in receptor number in the brain stem (PAG).

FIG. 13. Brain sections from rats treated chronically for 7 days with saline or morphine at 25 or 75 nmoles/hr were prepared as described in Example 9.E. To examine MOR-G protein coupling in these section, a [³⁵S]-GTPγS binding assay in response to morphine or DAMGO was carried out using slides containing sections from the midbrain, forebrain, and brain stem as described in Example 9.E. Brain areas in autoradiograms were quantitated using NIH Image software. Percent stimulation was calculated from optical densities (OD) according to the following equation:

Percent stimulation=(stimulated OD−basal OD)/basal OD×100%.

FIG. 13 is a histogram showing the results of this study for different brain regions: the striatum, the nucleus accumbens (NAc), the hippocampus, the thalamus, the amygdala, and the brain stem (PAG). The top panel (13.A.) shows morphine-stimulation of GTPγS binding, and the bottom panel (13.B.) shows DAMGO stimulation of GTPγS binding. Results are shown for rats treated for 7 days with saline (naïve) or 25 or 75 nmoles/hr morphine (MS 25 nmol and MS 75 nmol, respectively). Chronic morphine treatment sufficient to induce tolerance does not result in MOR-G protein uncoupling in the midbrain. There is a significant (P<0.05) reduction in MOR-G protein coupling in the brain stem (PAG). Chronic DAMGO treatment is associated with a reduction MOR-G protein coupling in the brainstem (P<0.001) and in the thalamus (P<0.05).

FIG. 14. To examine MOR distribution after acute and chronic treatment with morphine as compared to DAMGO, rats were implanted with i.c.v. cannulae. Morphine (MS, 50 nmoles) or DAMGO (DG, 1 nmole) was given directly via cannula. For the acute treatment, animals were sacrificed 30 mins following the first injection. For the chronic treatment, drugs were given twice a day: morning and afternoon, in 5 μl volume for 5 days, and the animals were sacrificed 30 mins following the final injection. Rats were deeply anesthetized with halothane and perfused with 4% paraformaldehyde in 0.1 M phosphate buffer. The brains were dissected out and post-fixed overnight in the same fixative and then transferred to a 30% sucrose buffer. Coronal sections (30 um thick) were cut on cryostat at −18° C., preincubated in PBT solution (0.1 M phosphate buffer, 2% BSA, and 0.2% Triton X-100) for 30 min, blocked in 5% normal goat serum in PBT solution for another 30 min, and then incubated with a rabbit anti-mu opioid receptor antibody at 1:5000 and mouse and NeuN antibody (which recognizes the neuronal-specific protein NeuN) at 1:5000 overnight at 4° C. The sections were washed several times with PBT and incubated in Alexa Fluor 488 goat anti rabbit antibody for mu-opioid receptor (green) and Alexa Fluor 546 goat anti mouse antibody for NeuN (red) for 2 hours at room temperature. The sections were then washed and mounted onto slides. The mu-opioid receptors and NeuN were visualized using a Zeiss confocal microscope with a 60× oil immersion objective.

FIG. 14.A. shows MOR distribution (green) for three brain regions, the striatum, the globus pallidus, and the ventral tegumental area, after acute treatement with saline, morphine, or DAMGO. NeuN distribution (red) indicates the location of the neuronal-specific protein NeuN. FIG. 14.B. shows MOR (green) and NeuN distribution (red) for the same regions after chronic treatment with saline, morphine, or DAMGO. MOR endocytosis is indicated by an increase in the green signal within the cell boundaries (which are stained more intensely green).

### DETAILED DESCRIPTION

Because of their importance in mediating the physiological actions of the majority of known neurotransmitters and hormones, G-protein coupled receptors (GPCR) are major targets for drug development. Many drugs have already been developed that target a GPCR. Both GPCR-agonists and antagonists can have useful therapeutic properties. Some examples of drugs that target a GPCR include, Atenolol (Tenormin®), a b1-adrenergic antagonist; Albuterol (Ventolin®), a b2-adrenergic agonist; Ranitidine (Zantac®), a H2-histamine antagonist; Loratadine (Claritin®), a H1-histamine antagonist; Hydrocodone (Vicodin®), a mu opioid agonist; Theophylline (TheoDur®), an adenosine antagonist; and Fluoxetine (Prozac®), an indirect-acting serotonin agonist.

Unfortunately, tolerance to GPCR-targetting drugs may develop from repeated or continuous use which renders the drug less useful. In some cases, physical dependence on the drug may also occur. As used herein “tolerance” means a decrease, usually a significant decrease, in the pharmacological effect of the drug at a particular dose following prolonged administration. Tolerance can also be manifested as a requirement for administration of higher and higher doses of a drug in order to achieve a comparable pharmacological effect. “Physical dependence” as used herein means a requirement for continued administration of increasing doses of the drug in order to prevent the development of symptoms associated with withdrawal of the drug. By “withdrawal” is meant physical symptoms of discomfort that are associated with, and attributable to, discontinuance of administration of a drug and can be alleviated by readministration of the drug.

The molecular basis by which such drug tolerance develops is the subject of debate. One widely-accepted current theory is that tolerance develops as a result of the loss of functional receptors at the cell surface by desensitization (by uncoupling of the receptor from the G-protein) and/or by endocytosis and down regulation of the receptor.

Another theory that has been recently put forward (Whistler et al. (1999) Neuron 23:737-746; Finn A. K. et al. (2001) Neuron 32:829-839) is that tolerance to a drug may develop as a cellular adaptive response to continued signalling from activated receptors at the cell surface, particularly where the receptor does not undergo drug-mediated endocytosis. In this view, endocytosis serves a protective role against the development of tolerance to the drug. Certain drugs, for instance morphine, are able to subvert the normal endocytosis processes for agonist-activated receptors, leading to prolonged cell surface signalling by the activated receptor. The cells responds to the prolonged signalling by making compensating changes downstream in the pathway to dampen the signal. Thus, higher and higher concentrations of agonist are required to produce the equivalent effect. Continued research will reveal whether these theories are correct, and in fact these theories may describe alternate possible pathways by which tolerance develops.

Regardless of the theoretical possibilities for the development of drug tolerance, the present inventors have now discovered that the development of tolerance to certain GPCR-activating drugs can be reduced, prevented or delayed by promoting the endocytosis of the receptor targetted by the drug. Endocytosis of the drug-activated receptor can be promoted in vivo by the co-administration, with the drug, of an agonist for the targetted receptor, where the agonist is one that promotes receptor endocytosis. Without being held to any particular theory, the agonist is believed to promote endocytosis of the drug-activated receptor because the G protein-coupled receptor dimerizes or oligomerizes in vivo, forming receptor complexes containing both the drug-activated receptors and the agonist-activated receptors. When the agonist promotes the endocytosis of the receptor to which it is bound, other receptors that are part of the oligomeric complex, including the drug-activated receptors, are “dragged” into the endosome along with the agonist-activated receptor. The drug-activated receptors can thus be resensitized and recycled to the cell membrane.

Treatment Methods

The present invention thus provides a method for reducing, preventing or delaying the development of tolerance to, and/or physical dependence on, a drug that activates a GPCR, wherein the tolerance and/or physical dependence develops as a consequence of the failure of the drug to promote endocytosis and resensitization of the activated GPCR, by co-administering, with the drug, an agonist for the GPCR, wherein the agonist promotes the endocytosis and resensitization of the GPCR, wherein the agonist is co-administered in an amount sufficient to promote endocytosis.

The method of the present invention will be useful for reducing, preventing or delaying the development of drug tolerance and/or physical dependence for drugs that activate a GPCR. By “drug” is meant any compound that is or can be used as a pharmaceutical. As used herein, “drug” will refer to drugs that target a GPCR. By “target a GPCR” by a drug is meant that the activity of the GPCR is affected by administration of the drug. The drug may interact directly or indirectly with the receptor and may activate or block the action of the receptor. Most of the description herein will pertain particularly to drugs that activate a GPCR, although the methods of the present invention are equally applicable to drugs that block the action of the receptor (“blockers”). By “activate a GPCR” by a drug is meant that the drug binds to the receptor, directly or indirectly, and causes the coupling of the receptor to G-protein, an initial step in the complex process of signalling from the receptor to intracellular effectors. The drug will thus act as an agonist of the GPCR. The method will be most suitable for use in connection with a drug, such as morphine, that does not promote the endocytosis of its receptor target. Such drugs are particularly liable for the development of tolerance and/or physical dependence. Whether a drug promotes endocytosis of its receptor target can be readily determined by techniques that are well known, for example, such as described Whistler et al. 1999. Assays for the recruitment of β-arrestin to the cell membrane in response to ligand binding, such as that described in U.S. Pat. No. 5,891,646, can also be used to determine if a particular drug promotes endocytosis of its receptor target or not. Other drugs that activate a GPCR target but do not promote receptor endocytosis include, for example, buprenorphine and delta9-tetrahydrocannabinol (THC).

The GPCR target of the drug may be any known GPCR or may be a GPCR that is identified by techniques that are well known in the art and described herein. The GPCR can be from any of the known families of GPCRs, including Family 1, Family 2, Family 3, Family 4, Family 5 and the cAMP Family (see Bockaert and Pin, supra, for a description of the various families). In most cases, the GPCR will be from Family 1, Family 2 or Family 3. G-protein coupled receptors vary in the particular trimeric GTP-binding protein (the “G-protein”) to which they couple. Numerous G-protein families are known that are generally distinguished by their sensitivities to various bacterial toxins (e.g., cholera toxin, pertussis toxin). For a review of G-proteins, see, Stryer and Bourne 1986 Annu. Rev. Cell. Biol. 2:391; Bourne and Stryer, 1992 358:541; Marinissen and Gutkind 2001 Trends Pharmacol. Sci 22:368. Some well known G-protein subtypes include Gₛ, G_{olf}, Gᵢ, Gₒ, Gₜ, G_{q}, G₁₁, G₁₂, G₁₃, G₁₄, G₁₅ and G_{z}. The GPCR drug target can be one that couples to any G-protein type. Preferably, the GPCR drug target will be a GPCR that couples to a G-protein of subtype Gᵢ, Gₒ, or G_{z}.

Techniques for identifying GPCRs are well known and include homology cloning, in which low stringency hydridization with known GPCR genes or cDNAs, or low homology sequence comparisons in human sequence databases, is used to identify related sequences, and expression cloning, in which cDNA libraries are screened for ligand-binding or cell-activation properties.

For use in connection with the present methods, the GPCR drug targets that are suitable are those GPCRs that are known to be, or are determined to be, resensitized and recycled to the cell membrane following agonist-mediated endocytosis, such as mu opioid receptor (MOR), rather than those receptors, such as delta opioid receptor (DOR), that are generally degraded following endocytosis. Whether a particular receptor follows the “recycling” pathway or the “degradation” pathway following agonist-mediated endocytosis, is known for many receptors and can be readily determined by methods that are well known in the art, such as those described in Whistler et al. 2001 J. Biol. Chem. 276:34331. Suitable GPCR drug targets in this regard include, but are not limited to, opioid receptors, serotonin receptors, dopamine receptors, neurokinin receptors, NPY receptors, adrenergic receptors, muscarinic receptors, chemokine receptors, metabotropic glutamate receptors, cannabinoid receptors, angiotensin receptors, somatostatin receptors, vasopression receptors prostaglandin receptors, histamine receptors, imidazoline receptors and GABA B receptors; particularly suitable are the opioid receptors, more particularly, the mu opioid receptor.

In addition, the GPCR drug target is one that normally forms a complex in vivo (either dimer or, preferably, oligomer) with other GPCRs of the same type. Many G-protein coupled receptors have been shown to dimerize or oligomerize, for example, opioid receptors, serotonin receptors, dopamine receptors, beta2-adrenergic receptor, somatostatin receptors and GABA B receptors. The formation of dimer or oligomer complexes in vivo is thought to be a general phenomenon for most, if not all, GPCRs. It is not clear if such receptor complex formation is associated with, or dependent upon, ligand binding, but for the purposes of the present invention, it is not important as receptor ligands (in the form of the drug and the agonist, at least) will necessarily be present. The GPCR may also form heteromeric complexes (that is, complexes with GPCRs of a different type). Whether such heteromeric complexes form in vivo is not completely understood although there is some evidence that suggests that they do (Devi, 2001). In the case of heteromeric complex formation of the GPCR drug target receptor, it will be apparent that agonists that bind to GPCRs other than the GPCR drug target receptor can be used. In the case of heteromeric complex formation, an agonist that binds to any of the GPCR types involved in the heteromeric complex could promote endocytosis of the entire complex and/or any of the complexed receptors. Preferably, for the present methods, the agonist will be one that binds to the same GPCR type as does the drug.

The methods of the present invention for reducing, preventing or delaying the development of drug tolerance, for treating pain and others described herein, comprise co-administering an agonist of the particular GPCR target of the drug or analgesic. By “agonist” is intended a compound that binds to and activates the GPCR. The term “agonist” will include partial agonists as well as full agonists, but does not include inverse agonists. It will be appreciated that, in this sense, the drug may also be an “agonist,” but for purposes of the present invention, the “agonist” will be other than the drug that is the subject of the method. Thus, if tolerance to, and/or physical dependence on, the drug morphine is the subject of the method, the agonist used will be other than morphine, even though morphine is a MOR agonist. For use in the present invention, the agonist will preferably bind to and activate the same receptors as those targetted by the drug. Thus, where it is desired to affect the development of tolerance to, and/or physical dependence on, for instance, morphine, an agonist that activates the mu opioid receptor (the morphine target receptor) will be used. Suitable agonists are those which promote endocytosis of their target receptors. In general, many agonists will promote the endocytosis of their target receptors. Whether a particular agonist promotes endocytosis of the receptor can be readily determined by methods that are well known in the art, as have been described herein with respect to the ability of the drug to promote endocytosis of its target GPCR. Suitable methods are described in Whistler et al 1999 and US Pat. No. 5,891,646. By “promotes endocytosis” is meant that agonist binding to the receptor is a triggering event for internalization (endocytosis) of the receptor. Following endocytosis, the receptor can be recycled to the cell membrane (resensitization) where it once again becomes available for ligand binding. The agonist may be selective or non-selective for the particular GPCR drug target, that is, the agonist may bind other receptors in addition to the drug target receptor. Preferably, the agonist will be selective for the drug target receptor. By “selective” is meant that the agonist binds to the drug target receptor with a higher affinity than to other GPCRs, preferably with at least a two-fold higher affinity than to other types of GPCRs.

The methods of the present invention are useful for reducing, preventing or delaying the development of drug tolerance and/or physical dependence. By preventing the development of drug tolerance and/or physical dependence is meant that no substantial tolerance to, and/or physical dependence on, the drug is seen over the typical course of treatment with the drug. By delaying the development of drug tolerance and/or physical dependence is meant that tolerance and/or physical dependence develops at a later time point than is usual during the typical course of treatment with the drug. By reducing drug tolerance and/or physical dependence is meant that drug tolerance and/or physical dependence develops in a smaller percentage of patients treated with the drug than is typically the case when the drug is administered alone.

In the methods of the present invention, the GPCR-activating drug is co-administered with a receptor agonist. By “co-administered” is meant that the drug and the agonist are present at the same time in the patient to be treated. The agonist need not be co-extensively present with the drug however. Thus, the agonist may be administered before the drug is administered, after the drug is administered, and/or simultaneously with the drug, provided that for some period of time the agonist and the drug are present together in the patient. For example, the drug may be administered continuously by i.v. over a period of several hours or days and the agonist may be administered intermittantly (e.g., once an hour, once a day, etc) over the same time period. Or the drug and the agonist may be administered together continuously, or both agonist and drug may be administered intermittantly at different times over the course of several hours or days, provided that, in this last example, the drug and the agonist will be present together for some period of time in the patient. Preferably, the drug and the agonist will be administered simultaneously, most preferably as a single composition. The drug will typically be administered in accordance with standard practices for the particular drug and indication.

The amount of agonist that will be co-administered with the drug will be sufficient to promote receptor endocytosis when administered in combination with the drug. This amount of agonist may or may not be sufficient to promote endocytosis when administered alone. It will be appreciated that a lesser amount of agonist may be sufficient to promote endocytosis of the receptor when the drug is also present as a threshhold of receptor occupancy by ligand (either drug or agonist) may be required to achieve endocytosis. The amount of agonist that will be sufficient can be readily determined by one of ordinanry skill in the art using the disclosure herein and methods that are well known in the art. In general, where the agonist is also a therapeutic, the amount of agonist co-administered will typically be no more than the amount required for the therapeutic effect and preferably the amount will be less than the amount required for the therapeutic effect. For the practice of the present methods, it is not necessary that the agonist be present in sufficient amount to produce any physiological or pharmacological effect of its own other than to promote endocytosis of the receptor. In general, this amount of agonist will be significantly lower than the amount required for a therapeutic effect. Because the drug and the agonist bind to the same GPCRs, the agonist will typically be co-administered in an amount that is significantly less than the amount of drug. In this way, most of the available target receptors will be occupied with the drug. It is only necessary for the agonist to bind to a small number of the target receptors in order to promote endocytosis of large numbers of receptors occupied by the drug because of the existence of the receptor complexes and the “dragging” phenomenon. Typically, the agonist need bind only between one receptor in 10 and 1 receptor in 10,000. More usually, the agonist need bind only between 1 in 10 and 1 and 100 receptors. The amount of agonist co-administered is generally less than the amount of drug and may be between about 10 and 10⁸ fold less (on a mole basis) than the amount of the drug (that is to say, that the drug is administered at between about 10-fold and 10⁸-fold greater amount than the agonist). Preferably, the amount of agonist co-administered is between about 10²-fold and 10⁶-fold less than the amount of the drug; more preferably between 10³-fold and 10⁵-fold less than the amount of the drug. It is desirable that the amount of agonist that is co-administered be as low as possible to minimize any side effects attributable to the activity of the agonist, while still being an amount of agonist sufficient to promote receptor endocytosis. In general, for both the drug and the agonist, the determination of suitable dosing regimens are within the competence of one of ordinary skill in the medical arts and may be found with reference to manufacturer's or supplier's instructions, or The Physician's Desk Reference.

In one aspect, the present invention provides a method for reducing, preventing or delaying the development of tolerance to, and/or physical dependence on, an opioid drug that activates the mu opioid receptor (MOR) but does not promote endocytosis of MOR. In this method, a MOR agonist that promotes MOR endocytosis is co-administered with the opioid drug to affect the development of tolerance to, and/or physical dependence on, the drug. By “opioid drug” is meant a drug whose receptor target is an opioid receptor. Suitable opioid drugs will be ones that, like morphine, do not promote endocytosis of the opioid receptor and are thus peculiarly liable for the development of tolerance and/or physical dependence. Preferably the agonist will be a selective mu opioid receptor agonist. Suitable agonists for this method include enkephalin, DAMGO, methadone, fentanyl, sufentanil, remi-fentanyl, etonitazene etorphine, and dihydroetorphine. Preferably, the agonist will be selected from methadone, fentanyl, sufentanil, remi-fentanyl, or etonitazene.

In another aspect, the present invention provides a method for reducing, preventing or delaying the development of tolerance to, and/or physical dependence on, morphine by co-administering a mu opioid agonist. Preferred mu opioid agonists include enkephalin, DAMGO, methadone, fentanyl, sufentanil, remi-fentanyl, etonitazene etorphine, and dihydroetorphine. More preferred agonists include methadone, fentanyl, sufentanil, remi-fentanyl, and etonitazene. “Morphine” includes (5α,6α)-7,8-didehydro-4,5-epoxy-17-methylmorphinan-3,6-diol and various derivatives, salts, hydrates, and solvates, that are useful as analgesics, including morphine hydrobromide, morphine hydrochloride, morphine methylbromide, morphine mucate, morphine oleate, morphine N-oxide, and morphine sulfate. Morphine derivatives include without limitation, normorphine and buprenorphine.

In this aspect of the invention, the morphine is administered in an analgesic amount by any conventional dosing regimen. For example, morphine may be administered at a dose of about 4 to about 8 mg iv, about 5 to about 12 mg im, or about 15 to about 60 mg po, typically about every 4 to 6 hours. Morphine may also be administered in a sustained release form for essentially continuous administration. The agonist, which may also be an analgesic, may be administered in an analgesic or a sub-analgesic amount. Preferably, if the agonist is an analgesic, the agonist will be administered in a sub-analgesic amount. The amount of agonist co-administered will be sufficient to promote mu opioid receptor endocytosis in the presence of the analgesic amount of morphine.

As used herein, an “analgesic” amount is the amount of the drug, for instance, morphine, which causes analgesia in a subject, and includes standard doses of the drug which are typically administered to cause analgesia (e.g, mg doses). A “sub-analgesic” amount of an agonist or drug is an amount less than the amount which causes analgesia in a subject, if administered in the absence of any other analgesic compound.

The present invention also provides a method for treating pain by co-administering morphine with a mu opioid agonist, wherein the agonist promotes the endocytosis of the MOR as described herein. The method provides an advantage over the administration of morphine alone in that the co-administration of an agonist reduces, prevents or delays the development of tolerance to, and/or physical dependence on, morphine that often accompanies prolonged use of this analgesic. For the treatment of pain, morphine will be administered in an analgesic amount, typically at a dose of about 4 to about 8 mg iv, about 5 to about 12 mg im, or about 15 to about 60 mg po, typically about every 4 to 6 hours. Other possible dosing regimens are within the competence of one of ordinary skill in the medical arts to determine and may be found with reference to manufacturer's or supplier's instructions, or The Physician's Desk Reference. The agonist will be administered in an amount sufficient to promote the endocytosis of the MOR in the presence of an analgesic amount of morphine. As the co-administration of morphine with the agonist will moderate the tolerance typically observed for morphine use, the analgesic amount of morphine may be less than the amount that would be standardly administered to treat pain.

It will be appreciated that the methods of the present invention for reducing, preventing or delaying the development of tolerance to a drug will also be useful for reducing, preventing or delaying the development of withdrawal. One possible mechanism for the development of drug tolerance, as explained herein, links the development of tolerance to the occurrence of certain adaptive cellular changes, such as superactivation of the cAMP pathway. These cellular changes are not readily reversible in the absence of the drug and are believed to play a role in the phenomenon of “withdrawal” often associated with the discontinuance of drug administration. If tolerance to the drug is not allowed to develop, the cellular adaptive changes underlying the tolerance phenomenon will necessarily not occur. Thus, methods for reducing, preventing or delaying the development of tolerance will also reduce, prevent or delay the occurrence of withdrawal as well.

The agonists and drugs for use in the present invention may be in, but are not limited to, the form of free bases or pharmaceutically acceptable acid addition salts thereof, or free acids or esters or anhydrides thereof. Examples of suitable acids for salt formation include but are not limited to methanesulfonic, sulfuric, hydrochloric, glucuronic, phosphoric, acetic, citric, lactic, ascorbic, maleic, and the like.

The agonist or the drug may be administered to a human or animal subject by known procedures including but not limited to oral, sublingual, intramuscular, subcutaneous, intravenous, and transdermal modes of administration. Preferably, the agonist and the drug will be administered intravenously. When a combination of these compounds are administered, they may be administered together in the same composition, or may be administered in separate compositions. If the agonist and the drug are administered in separate compositions, they may be administered by similar or different modes of administration, and may be administered simultaneously with one another, or shortly before or after the other.

The agonist and the drugs may be formulated in compositions with a pharmaceutically acceptable carrier. The carrier must be “acceptable” in the sense of being compatible with the other ingredients of the formulation and not deleterious to the recipient thereof. Examples of suitable pharmaceutical carriers include lactose, sucrose, starch, talc, magnesium stearate, crystalline cellulose, methyl cellulose, carboxymethyl cellulose, glycerin, sodium alginate, gum arabic, powders, saline, water, among others. The formulations may conveniently be presented in unit dosage and may be prepared by methods well-known in the pharmaceutical art, by bringing the active compound(s) into association with a carrier or diluent, as a suspension or solution, and optionally one or more accessory ingredients, e.g. buffers, flavoring agents, surface active agents, and the like. The choice of carrier will depend upon the route of administration.

For intravenous, intramuscular, or subcutaneous administration, the compounds may combined with a sterile aqueous solution which is preferably isotonic with the blood of the recipient. Such formulations may be prepared by dissolving solid active ingredient in water containing physiologically compatible substances such as sodium chloride, glycine, and the like, and having a buffered pH compatible with physiological conditions to produce an aqueous solution, and rendering the solution sterile. The formulations may be present in unit or multi-dose containers such as sealed ampoules or vials.

For transdermal administration, the compounds may be combined with skin penetration enhancers such as propylene glycol, polyethylene glycol, isopropanol, ethanol, oleic acid, N-methylpyrrolidone, and the like, which increase the permeability of the skin to the compounds, and permit the compounds to penetrate through the skin and into the bloodstream. The compound/enhancer compositions also may be combined additionally with a polymeric substance such as ethylcellulose, hydroxypropyl cellulose, ethylene/vinylacetate, polyvinyl pyrrolidone, and the like, to provide the composition in gel form, which can be dissolved in solvent such as methylene chloride, evaporated to the desired viscosity, and then applied to backing material to provide a patch.

Compositions

The present invention provides compositions useful in the methods of the present invention. The compositions comprise a drug that targets a GPCR and an agonist for the same GPCR. The drug and the agonist do not comprise the same compound. In particular, the drug is a compound that does not promote substantial endocytosis and resensitization of the targetted GPCR, whereas the agonist is a compound that does promote such endocytosis and resensitization. In one embodiment, the composition comprises a drug that activates a GPCR and an agonist for the GPCR. Exemplary drugs and agonists suitable for use in the compositions of the invention include those described above in connection with the treatment methods of the invention. In a preferred embodiment, a composition of the invention includes, in addition to a drug and agonist, a pharmaceutically acceptable carrier, such as, for example, those described above.

The agonist is present in the composition in an amount sufficient to promote endocytosis and resensitization of the targetted GPCR. The agonist is generally present in the composition in an amount that is less than the amount of the drug. Typically, the agonist is present in an amount that is between about 10 and about 10⁸-fold less (on a mole basis) than the amount of the drug (that is to say, that the drug is present at between about 10-fold and about 10⁸-fold greater amount than the agonist). Preferably, the amount of agonist present in the composition is between about 10²-fold and about 10⁶-fold less than the amount of the drug; more preferably, between about 10³-fold and about 10⁵-fold less.

In a preferred embodiment, the drug includes an analgesic and is present in the composition in an analgesic amount (i.e., an amount that causes analgesia upon administration to a subject). In a variation of this embodiment, the agonist also includes an analgesic and is present in the composition in a sub-analgesic amount.

For compositions intended for in vivo applications, the doses of drug and agonist depend, for example, upon the therapeutic objectives, the route of administration, and the condition of the subject. Accordingly, it is necessary for the clinician to titer the dosage and modify the route of administration as required to obtain the optimal therapeutic effect. Generally, the clinician begins with a low dose and increases the dosage until the desired therapeutic effect is achieved. Suitable starting doses for a given drug or agonist are known or can be extrapolated from in vitro and in vivo data, such as that described in the examples below.

A preferred composition of the invention comprises an opioid drug that targets the mu opioid receptor, and the agonist comprises a mu opioid receptor agonist. In a variation of this embodiment, the opioid drug activates the mu opioid receptor. In a composition of the invention useful for treating pain, the opioid drug includes an analgesic and is present in the composition in an analgesic amount. In a preferred variation of this embodiment, the mu opioid receptor agonist also includes an analgesic, but is present in the composition in a sub-analgesic amount.

Exemplary compositions of the invention include morphine and an agonist for the mu opioid receptor other than morphine. Preferred compositions comprise morphine and one or more agonists selected from DAMGO, methadone, fentanyl, sufentanil, remifentanyl, etonitazene, and etorphine, in addition to the mu opioid receptor agonists described above.

Preferably, where appropriate for the administration of the composition, the drug and the agonist are present in an admixture in a single container.

Kits

The present invention also provides kits including: (1) a drug that targets a GPCR and (2) an agonist for the same GPCR in separate containers. The considerations for selecting and formulating the drug and agonist (i.e., suitable carriers, doses, etc.) are the same as described above for compositions of the invention. Suitable containers for a given application are well known in the art.

In a preferred embodiment, a kit of the invention includes instructions for performing a method of the invention. While the instructional materials typically comprise written or printed materials they are not limited to such. Any medium capable of storing such instructions and communicating them to an end user is contemplated by this invention. Such media include, but are not limited to, electronic storage media (e.g., magnetic discs, tapes, cartridges, chips), optical media (e.g., CD ROM), and the like. Such media can include addresses to internet sites that provide such instructional materials.

Screening Methods

The present invention additionally provides a method of screening for an agent that reduces, prevents or delays the development of tolerance to, and/or physical dependence on, a drug that targets a GPCR. The method entails contacting a test agent with a cell comprising the GPCR, determining whether the test agent promotes the endocytosis of the GPCR, and selecting a test agent that promotes such endocytosis as an agent that may reduce, prevent or delay the development of tolerance to, and/or physical dependence on, the drug.

Cells useful in the screening methods of the invention either express a suitable endogenous GPCR or can be engineered to express a heterologous GPCR using standard recombinant techniques. Endocytosis is measured by contacting the cell with sufficient test agent to bind the G protein-coupled receptor. Endocytosis is then determined in the presence and absence (or presence of a lower amount) of test agent to determine whether the test agent promoted endocytosis. Preferably, the contacting step is carried out in vitro to facilitate the screening of large numbers of test agents.

Endocytosis can be determined by any of a variety of methods, including those described herein. For example, cell surface receptors can be measured and/or receptor proteolysis or localization in lysozomes can be determined. Alternatively, receptor downregulation can be determined indirectly by measuring desensitization of receptors after activation with an test agent. Desensitization can be determined, for example, by measuring a biological effect that is mediated by the receptor. Generally, it will be most convenient to measure cell surface receptors using a radio- or immunoassay. Briefly, cells expressing the cell surface receptor of interest are incubated with a suitable test agent under conditions designed to provide a saturating concentration of test agent over the incubation period. After test agent treatment, the cells are recovered and assayed for radioligand binding. Cells that form monolayers can, for example, be collected with phosphate-buffered saline (PBS) supplemented with EDTA, followed by washing four times by centrifugation with 10 mL of warm (37° C.) PBS and one time by centrifugation with 10 mL of Krebs-Ringer HEPES buffer (KHRB: 110 mM NaCl, 5 mM KCl, 1 mM MgCl₂, 1.8 mM CaCl₂, 25 mM glucose, 55 mM sucrose, 10 mM HEPES, pH 7.3). Radioligand binding can then be carried out in 120 μL of KHRB containing equal amounts of washed cells (50-100 μg of protein). Incubations can be carried out, for example, for 30 minutes at room temperature. Cells can then be harvested and washed using vacuum filtration on glass fiber filters, followed by a determination of the radioligand bound to the filters.

Exemplary immunoassays for determining receptor endocytosis are described below in Examples 2-5, 7, and 9.G.

In addition, high-throughput methods can be employed for the screening method of the invention. An exemplary assay amenable to high-throughput screening makes use of a pH sensitive dye (e.g., Cypher from Amersham Biosciences) that fluoresces only at low pH, such as the acidic environment of the endosome. In this assay, cells stably expressing N-terminally epitope-tagged MOR are incubated with an antibody specific for the epitope tag. The antibody is conjugated to the pH-sensitive dye. Endocytosis is measured by detecting the fluorescent signal from the labeled antibody which has bound to the epitope-tagged MOR and been endocytosed.

In preferred embodiments of the screening method of the invention, the test agent comprises an analgesic. In one exemplary embodiment, the GPCR is the mu opioid receptor, and the test agent(s) include one or more mu opioid receptor agonists.

Test Agent Database

In a preferred embodiment, generally involving the screening of a large number of test agents, the screening method includes the recordation of any test agent that promotes endocytosis of GPCR of interest in a database of agents that may reduce, prevent or delay the development of tolerance to, and/or physical dependence on, a drug that targets the GPCR.

The term “database” refers to a means for recording and retrieving information. In preferred embodiments, the database also provides means for sorting and/or searching the stored information. The database can employ any convenient medium including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems,” mainframe systems, distributed nodes on an inter- or intra-net, data or databases stored in specialized hardware (e.g. in microchips), and the like.

Test Agents Identified by Screening

When a test agent is found to promote endocytosis of GPCR of interest, a preferred screening method of the invention further includes combining the test agent with a carrier, preferably pharmaceutically acceptable carrier, such as are described above. Generally, the concentration of test agent is sufficient promote endocytosis and resensitization of the targetted GPCR when the composition is contacted with a cell, as described above for the drug and agonist-containing compositions and the treatment methods of the invention. This concentration will vary, depending on the particular test agent and specific application for which the composition is intended. As one skilled in the art appreciates, the considerations affecting the formulation of a test agent with a carrier are generally the same as described above.

Methods of the invention can also include combining resultant test agent compositions with a drug that targets a GPCR to produce compositions such as the drugagonist-containing compositions described above. As discussed above, suitable drugs include compounds that do not promote substantial endocytosis of the targetted GPCR and are preferably compounds that activate the GPCR. When the drug is one that activates the GPCR, the drug concentration will be sufficient to activate the G protein-coupled receptor when the composition is contacted with a cell containing a suitable receptor.

The following examples are provided by way of illustration and are not intended to limit the invention.

### EXAMPLES

### Experimental Procedures

Cell culture and immunocytochemistry. Human Embryonic Kidney (HEK) 293 cells (American Type Culture Collection) were grown in DMEM (Gibco BRL) supplemented with 10% Fetal Bovine Serum (Hyclone). Mu opioid receptor and CRE-Luciferase (Promega) constructs were stably transfected using calcium phosphate co-precipitation, with single colonies chosen and propagated in the presence of selection-containing media. For immunocytochemistry, cells were grown on poly-lysine coated coverslips and incubated with 3.5 μg/ml M1 anti-FLAG and/or 3.5 μg/ml HA-11 antibody (Covance) for 30 minutes. Cells were then treated with agonist as specified, fixed in 4% formaldehyde in PBS, permeabilized in 0.1% Triton X-100 in blotto, and stained. Cells stained for only one receptor type were stained with Texas red-conjugated Donkey anti-Mouse antibody (Jackson Immunoresearch). Cells that were stained for both FLAG and HA tagged receptors simultaneously were first incubated with rabbit anti-IgG_{2b} antibodies (Zymed) followed by staining with Texas red Donkey anti Mouse antibody (Jackson Immunoresearch) and FITC-conjugated Goat anti Mouse IgG₁, antibody (Boehringer). Images were acquired using a custom-configured inverted microscope (Prairie Systems, Madison, Wis.) with a Zeiss 63× oil objective, or a Zeiss confocal with a 60× oil objective.

Immunoprecipitation. Cells were grown to 80% confluency in 10 cm dishes and treated with 5 μM agonist for 30 minutes or left untreated. Cells were washed 2× in PBS and lysed in NDM lysis buffer (10 mM HEPES, pH 7.5, 150 mM NaCl, 2 mM MgCl, 1 mM CaCl, 0.5% n-dodecyl-β-D-maltoside). Lysate was cleared by centrifugation at 10,000 rpm for 10 minutes at 4° C., and cleared lysate was immunoprecipitated with 40 μl M2-conjugated sepharose (Covance) overnight at 4° C. Immunoprecipitates were extensively washed with NDM buffer followed by 2 washes with 10 mM Tris, pH 7.5. Receptors were deglycosylated with PNGase (NEB) in 10 mM Tris pH 7.5 for 2 hours at 37° C., denatured with SDS sample buffer and resolved by SDS-PAGE. Blots were blocked in Blotto, incubated with a biotinylated M2 anti FLAG antibody (1:250, Covance) for 2 hours and developed with streptavidin overlay using ABC reagents (Vector laboratories) and ECL reagents (Amersham) as a control, or incubated with HA-11 antibody (1:1000 Covance) for 2 hours and HRP-conjugated Goat anti mouse (1:3000, Jackson Immunoresearch) for 1 hour and developed with ECL reagents to detect oligomers.

CRE-luciferase reporter expression assays. Cells were grown to confluency in 24 well plates. For acute experiments, cells were given drug for 4 hours and the fold inhibition of forskolin-stimulated luciferase activity measured. For chronic treatment experiments cells were given drug for 14 hours, rinsed 3 times in drug-free media to initiate a withdrawal phase, then given 2 μM forskolin for the 4 hour withdrawal phase, and luciferase activity measured. 14 hours was chosen after an initial time course of morphine-induced superactivation in MOR-expressing HEK293 cells demonstrated that superactivation at this time point was highly reproducible. For all treatment conditions, cells were rinsed once in PBS immediately prior to luciferase measurement. 100 μl Cell Culture Lysis Reagent (Promega) was added to each well, a 20 μl cell lysate aliquot was transferred to an opaque 96 well plate, 100 μl substrate added per well using a Lucy 2 luminometer (Anthos), and light measurements collected. Data were exported to Microsoft Excel for compilation, and GraphPad Prism 3.0 for graphical display, non-linear regression curve fitting, and subsequent statistical analyses.

Animals. Male Sprague-Dawley rats (250-300 g, Simonsen Laboratories, Inc. Gilroy, Calif.) were housed individually in temperature-controlled rooms with a 12-hr light/dark cycle. Food and water were available ad libitum. All procedures used in this study were in agreement with the NIH Guide for the Care and Use of Laboratory Animals and were approved by the Animal Care and Use Committee at Gallo Center of the University of California, San Francisco.

Preparation and implantation of intrathecal (IT) catheters. Catheter implantation was performed according to methods of Yaksh with minor modifications (Yaksh and Rudy, 1976 Physiol. Behav. 17:1031; Yaksh and Stevens, 1986 Pharmacol. Biochem. Behav. 25: 483). Two types of catheters were prepared depending on the regimen for test drug delivery. For the morphine alone groups (and the saline only controls), a 3-cm length of polyethylene tubing, PE-60, was connected to an 8-cm length of PE-10 tubing by heating. For the chronic morphine+DAMGO/saline groups, a Y-shape catheter was prepared. For catheter implantation, rats were anesthetized with isoflurane and placed on a stereotaxic device with the head flexed forward. The PE-10 catheter was inserted into the spinal subarachnoid space through an incision in the atlanto-occipital membrane and advanced caudally extending to the lumbar enlargement of the spinal cord. After implantation of IT catheters, rats were returned to their home cages and allowed 7 days to recover from surgery. Those rats with normal motor function were implanted with a subcutaneous mini-osmotic pump (Model 2001, DURECT Corp., Cupertino, Calif.), that had been prefilled with morphine or saline, on the dorsal part of neck under light isoflurane anesthesia.

Drug treatments. DAMGO and morphine sulfate were purchased from Sigma (St. Louis, Mo.) and dissolved in 0.9% physiological saline. The test drug was delivered via either single injection or chronic infusion. Morphine or saline was infused via mini-osmotic pump at a constant rate of 1 μl/hr. DAMGO or saline was injected through one arm of the Y-shape catheter at 15 μl volume.

Antinociception test. Rats were tested for antinociception using the radiant heat tail-flick procedure. The light intensity was adjusted to achieve base-line latencies of 1.5 to 2 seconds; a maximum latency of 6 seconds was set as the cut-off time to minimize damage to the tail. For the morphine alone group and the saline controls, the animals were tested by tail-flick once a day for 7 days following implantation of the mini-osmotic pump. For the morphine+DAMGO and morphine+saline groups, following mini pump implantation, rats were administrated DAMGO or saline via the other arm of the Y-shape catheter twice a day for 7 days at 9:00 AM and 4:30 PM. Antinociception was tested by tail-flick 30 min after the afternoon administration. The behavioral data of antinociception were compared and statistically analyzed by two-way analysis of variance followed by Bonferroni post-test, where P<0.05 was considered significant.

Immunohistochemistry. The rats were anesthetized with intramuscular ketamine hydrochloride (80 mg/kg) and xylazine hydrochloride (12 mg/kg) and perfused with 4% paraformaldehyde in 0.1 M phosphate buffer immediately following the tail-flick test at day 7 following pump implantation. The segment of spinal cord proximal to the tip of the catheter was dissected out, post-fixed overnight in the same fixative and then transferred to a 30% sucrose buffer solution. Sagittal sections (30 μm) were cut on a freezing microtome, preincubated in PBT solution (0.1 M phosphate buffer+0.2% BSA and 0.2% Triton X-100) for 30 min, blocked in 5% normal goat serum in PBT solution for another 30 min and then incubated in a rabbit anti-MOR antibody (DiaSorin, Stillwater, Minn.) at a 1:5000 dilution and mouse anti-NeuN antibody to identify the neurons in the section (Mullen R. J. et al. (1992) Development 116:201-211) (Chemicon International, Temecula, Calif.) at 1:300 overnight at 4° C. Sections were extensively washed with PBT and incubated in Cy-3-conjugated goat anti-rabbit antibody (Jackson ImmunoResearch, West Grove, Pa.) and FITC-conjugated goat anti-mouse antibody (Jackson ImmunoResearch, West Grove, Pa.) both at a 1:600 dilution for 2 hr at room temperature. The sections were then washed and mounted on slides. MOR distribution was examined with a Zeiss confocal microscope using a 60× oil immersion objective. For quantification, slides from at least two different rats for each condition were stained by one researcher and encoded and vesicles were counted blind by a second individual from the middle section of at least 8 cells per condition. Following compilation of vesicle counts, the code was broken.

### Example 1

Oligomerization of MOR with D MOR in Cell Culture.

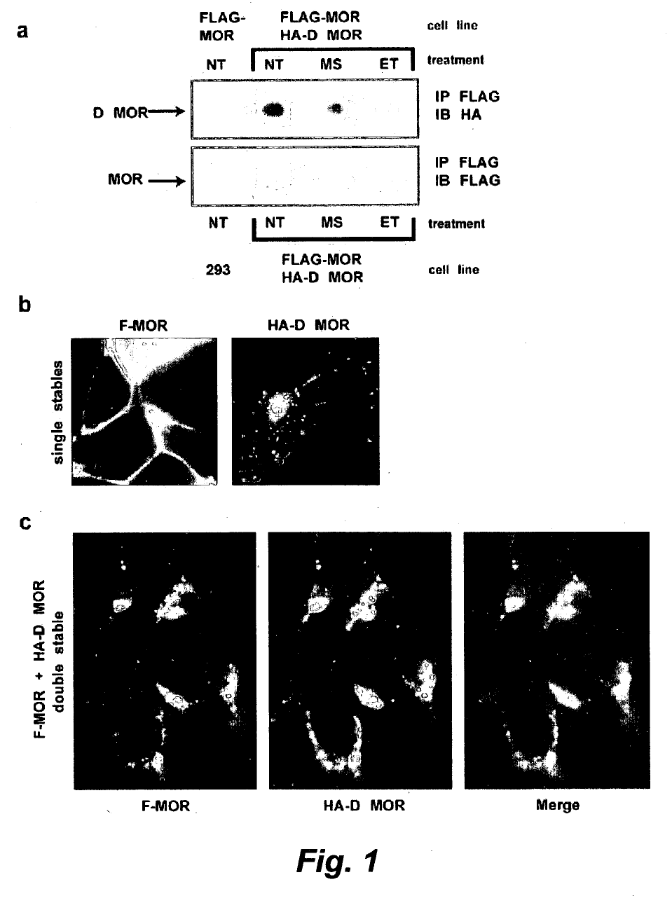

First we examined whether the wild type MOR could form heterodimers with a previously described mutant MOR, D MOR, that has altered trafficking properties (Finn A. K. et al. (2001) Neuron 32:829-839; Whistler J. L. et al. (1999) Neuron 23:737-746). The D MOR receptor is a chimera in which the cytoplasmic tail of the MOR has been replaced by the corresponding residues of the delta opioid receptor. This confers upon this receptor a gain-of-function phenotype whereby morphine can promote receptor phosphorylation, arrestin recruitment and endocytosis (Whistler J. L. et al. (1999) Neuron 23:737-746). Human embryonic kidney (HEK) 293 cell lines stably transfected with both a FLAG-tagged MOR and an HA-tagged D MOR were generated. Cells were treated with morphine (MS) or etorphine (ET) or left untreated (NT), and the FLAG tagged MORs were immunoprecipitated. Cells were permeabilized and receptors were immunoprecipitated with anti-FLAG antibodies, resolved by SDS-PAGE and transferred. Oligomers were detected by immunoblotting with antibodies directed against the HA tag of the D MOR receptor (FIG. 1A—upper). As a control, an aliquot of the immunoprecipitate was also immunoblotted with anti-FLAG antibodies (FIG. 1A—lower panel). Cells expressing only FLAG-MOR (FIG. 1A—upper panel, left lane) or no receptor (293, lower panel, left lane) were used as controls for antibody specificity. The D MOR receptor efficiently coimmunoprecipitated with the MOR. Dimerization did not appear to be ligand dependent.

### Example 2

Receptor Oligomerization Alters MOR Trafficking Properties.

We next assessed whether the heterodimerization of MOR and D MOR could affect the trafficking of the receptors using immunocytochemical methods. The double stable cell lines described in Example 1 were fed antibodies to the N-terminal extracellular tags of the FLAG-MOR and HA-D MOR. Cells were treated with morphine (5 μM, 30 minutes) and fixed. Control cell lines that expressed only one of the receptors, either FLAG-MOR or HA-D MOR, were treated analogously. All cells were then permeabilized and stained with fluorescently conjugated secondary antibodies. In this way, only receptors that were initially on the cell surface were detected. As expected, cells expressing only MOR failed to show significant receptor endocytosis when treated with morphine whereas the D MOR receptor efficiently internalized (FIG. 1B). In contrast, the MOR in the cell line that co-expressed D MOR underwent significant endocytosis in the presence of morphine with a substantial number of vesicles showing colocalization of both receptors (FIG. 1C). We called this phenomenon “dragging” because it appeared that the D MORs could drag the MORs into the cell in response to morphine, presumably because these receptors were making heterodimers.

### Example 3

The D MOR Affects MOR Trafficking in Cultured Neurons.

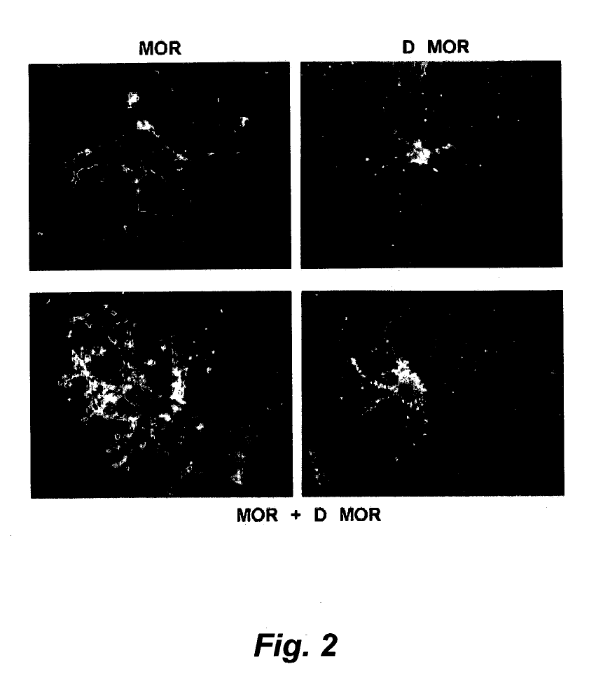

To ensure that this phenomenon was not an artifact of the HEK 293 cell model, we examined whether dragging also occurred in cultured neurons. Hippocampal neuron cultures were prepared from rat and were allowed to mature for three weeks. Cultures were then transfected with FLAG-MOR alone, HA-D MOR alone or both receptors. Cultures were fed anti-FLAG and/or anti-HA antibodies then treated for 30 minutes with 5 μM morphine. As previously reported (Whistler J. L. et al. (1999) Neuron 23:737-746), neurons expressing MOR alone expressed receptor primarily on the plasma membrane following morphine treatment (FIG. 2, upper left panel). In contrast, cells expressing D MOR alone showed efficient redistribution of receptors to endocytic vesicles following activation by morphine (FIG. 2 upper right panel). Importantly, in neurons that expressed both receptors, both the D MOR and the wild type MOR were redistributed to endocytic vesicles following activation by morphine (FIG. 2, lower panels). These results demonstrate that the D MOR receptor can drag the MOR into neurons.

### Example 4

DAMGO Facilitates Morphine-Induced Endocytosis of MOR.

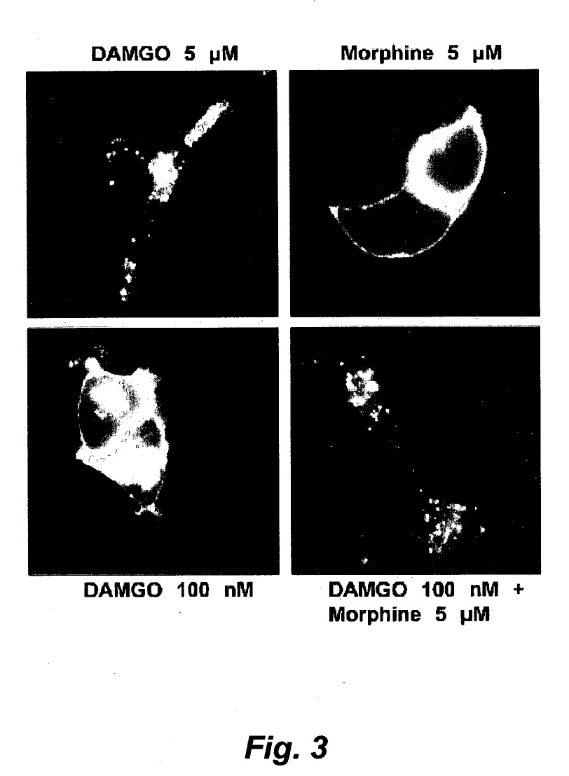

We have also observed that wild type MORs can homodimerize with one another just as they heterodimerize with D MOR (data not shown). Because of this observation, we designed an experiment to ask whether we could facilitate receptor dragging using the pharmacology of different MOR agonists. DAMGO, a hydrolysis resistant derivative of enkephalin, promotes robust endocytosis of the MOR (Keith D. E. et al. (1996) J Biol Chem 271:19021-19024) and has an affinity for the MOR similar to that of morphine (Raynor K. Kong et al. (1994) Mol Pharmacol 45:330-334). These observations allowed us to address whether a DAMGO-occupied MOR could drag a morphine-occupied MOR into the cell. HEK 293 cells expressing only wild type MOR were treated with a saturating dose of DAMGO (5 μM) or a saturating dose of morphine (5 μM). As expected, cells treated with DAMGO alone showed robust endocytosis of receptor (FIG. 3, upper left) while cells treated with morphine alone showed little endocytosis of receptor (FIG. 3, upper right). When we treated the same cells with a non-saturating dose of DAMGO (100 nM) there was significantly less receptor endocytosis (FIG. 3, lower left), presumably because of low receptor occupancy.

We next asked whether these few DAMGO-occupied receptors could drag morphine-occupied receptors into the cell. To accomplish this, we treated cells simultaneously with the non-saturating dose of DAMGO (100 nM) and a saturating dose of morphine (5 μM). Assuming all receptors are monomers, one would predict that the saturating dose of morphine would act as an antagonist for the sub-maximal endocytosis induced by the sub-saturating dose of DAMGO. Remarkably, cells treated in this way showed robust receptor endocytosis (FIG. 3, lower right). We attribute this phenomenon to the ability of a few DAMGO-activated receptors to drag several morphine activated receptors into the cell.

### Example 5

Activation of the Beta-2 Adrenergic Receptor (B2AR) does not Cause Endocytosis of Morphine-Activated MOR.

The results from Example 4 suggest that the mu opioid receptors are making oligomers rather than simple dimers and that a single DAMGO-occupied receptor in an oligomeric complex with other morphine-occupied receptors is sufficient to recruit the endocytic machinery and facilitate oligomer internalization. Alternatively, it might suggest that the few DAMGO-activated receptors in the cell are bringing a high local concentration of the endocytic machinery, in particular arrestin, to the morphine-activated receptors. We have demonstrated previously that overexpression of arrestin can facilitate morphine-induced endocytosis of wild type MOR (Whistler J. L. et al. (1998) Proc Natl Acad Sci USA 95:9914-9919). To differentiate between these possibilities, we examined whether an unrelated GPCR, the beta-2 adrenergic receptor (B2AR), when activated, could facilitate the heterologous endocytosis of the morphine-activated MOR.

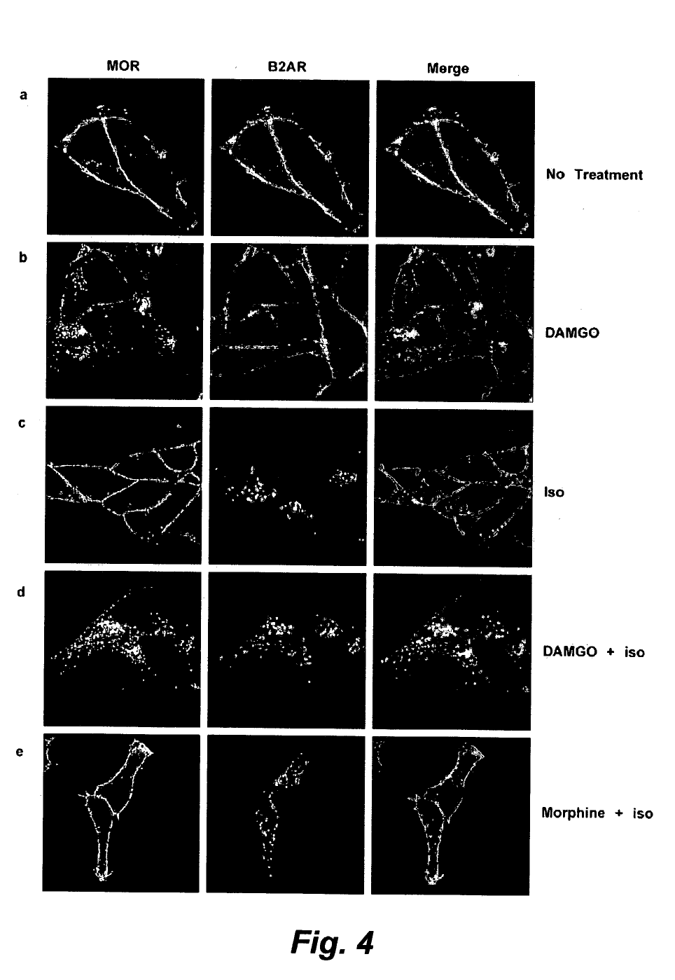

HEK 293 cells were generated that stably expressed both the FLAG-tagged MOR and an HA-tagged B2AR. The cells were incubated with antibodies to both epitope tags to label cell surface receptors, treated with various agonists or agonists combinations, then stained for both receptors. Cells were treated with DAMGO or isoproterenol (a B2AR agonist) or combinations of DAMGO and isoproterenol or morphine and isoproterenol. All treatments were for 30 minutes with 5 μM each agonist. Both receptors were expressed primarily on the cell surface in the absence of any agonist (FIG. 4A). As expected DAMGO promoted endocytosis of the MOR but not B2AR (FIG. 4B), while isoproterenol (iso) promoted endocytosis of B2AR receptor but not MOR (FIG. 4C). In the presence of both DAMGO and isoproterenol, both receptors were efficiently internalized (FIG. 4D). However, isoproterenol-activated B2ARs were not able to drag morphine-activated MORs into the cell (FIG. 4E). These results suggest that heterologous activation of the B2AR receptor and its consequent membrane recruitment of arrestin is insufficient to promote the endocytosis of nearby MORs. Hence it is likely that receptors must be in an oligomeric complex in order for dragging to be efficient.

### Example 6

DAMGO Reduces Morphine-Induced cAMP Superactivation.

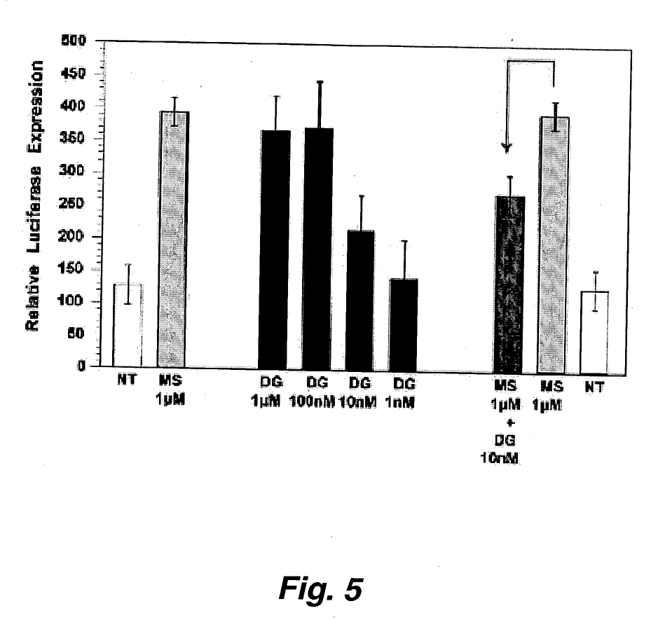

Chronic morphine treatment of animals, as well as cells in culture, produces a compensatory upregulation of the cAMP pathway (Sharma S. K. et al. (1975) Proc Natl Acad Sci USA 72:3092-3096; Bonci A. et al. (1997) J Neurosci 17:796-803; Avidor-Reiss T. et al. (1996) J Biol Chem 271:21309-21315), an effect that has been studied as a cellular hallmark of opiate withdrawal that we have also demonstrated contributes directly to a form of cellular tolerance (Finn A. K. et al. (2001) Neuron 32:829-839). Previously we have demonstrated that receptor endocytosis can reduce this compensatory upregulation (Finn A. K. et al. (2001) Neuron 32:829-839). Hence we predicted that receptor dragging could reduce superactivation in our cell culture model. We assessed the functional consequences of dragging using a previously described cell line that expresses MOR and a CRE-luciferase reporter gene (Finn A. K. et al. (2001) Neuron 32:829-839). Cells stably expressing MOR and a CRE-luciferase reporter gene were treated chronically (14 hours) with morphine (1 μM), DAMGO (1 μM, 100 nM, 10 nM or 1 nM), or both drugs (1 μM morphine+10 nM DAMGO) and superactivation of the cAMP pathway was assessed relative to untreated cells. Morphine (1 μM) caused pronounced superactivation (FIG. 5-“MS 1 μM”). DAMGO also caused superactivation in a dose dependent manner. A dose of DAMGO that caused little superactivation (FIG. 5-“DG 10 nM”) when administered alone, when administered concurrently with the superactivation-inducing dose of morphine (FIG. 5 “MS 1 μM+DG 10 nM”) reduced the morphine-induced superactivation. P<0.01, two-way ANOVA, Tukey's post test. DAMGO also induced superactivation in this cell line in a dose dependent manner (FIG. 5, black bars), despite its ability to promote receptor endocytosis. We attribute this to DAMGO's enhanced potency and hence greater numerator value in its RAVE (“RAVE” refers to relative activity versus endocytosis; see Whistler et al. 1999 for discussion of RAVE values) compared to that of morphine (Avidor-Reiss T. et al. (1996) J Biol Chem 271:21309-21315). Remarkably, a low dose of DAMGO (10 nM), which alone produced little superactivation, substantially reduced superactivation when it was administered simultaneously with a superactivation-inducing dose of morphine (1 μM).

Taken together, these data demonstrate that a second, endocytosis-promoting agonist can facilitate morphine-induced receptor endocytosis, consequently reducing the RAVE value of morphine, and reducing the compensatory adaptive cellular changes that lead to upregulation of the cAMP pathway, at least in a cell culture model. These observations lead us to design experiments (Example 7) to examine the role of receptor endocytosis in the development of tolerance in an intact animal.

### Example 7

DAMGO Facilitates Morphine-Induced Endocytosis in Rat Spinal Cord Neurons.

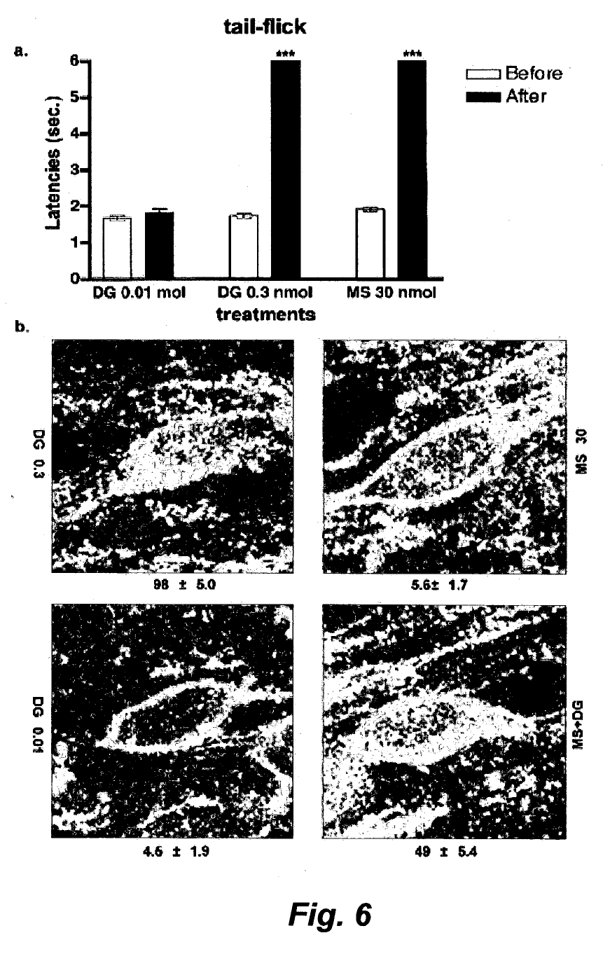

To begin these studies, we first assessed whether we could facilitate morphine-induced endocytosis of the MOR using a low dose of DAMGO in vivo. Rats (4-6 per group) were implanted with an intrathecal catheter through which either an acute injection of agonist could be given or chronic drug could be administered by an osmotic mini pump. We first examined the effects of a single acute injection of morphine and DAMGO on analgesia and endocytosis. Analgesia was assessed by testing tail flick latency before and 30 min after drug administration. 0.3 nmoles of DAMGO or 30 nmoles of morphine produced significant analgesia (*** p<0.001), whereas 0.01 nmoles of DAMGO had no analgesic effect (p>0.05) Student's t-test. Consistent with previous studies (Advokat C. (1993) Pharmacol Biochem Behav 45:871-879; Malmberg A. B. et al. (1992) J Pharmacol Exp Ther 263:264-275; Trafton J. A. et al. (2000) J Neurosci 20:8578-8584), an acute high dose of both DAMGO (0.3 nmoles) and morphine (30 nmoles) produced profound analgesia (FIG. 6A). Following the behavioral testing, these animals were perfused and the distribution of MORs was examined using immunohistochemical staining. Neurons from the lamina II of the spinal cord dorsal horn were examined because they play an important role in pain transmission (Yaksh T. L. (1999) Trends Pharmacol Sci 20:329-337). MORs were detected in numerous endosomes throughout the cell body of the lamina II neurons of rats treated with 0.3 nmoles DAMGO (FIG. 6b, upper left panel) indicative of pronounced receptor endocytosis. In contrast, the MORs in lamina II neurons of the rats treated with the equi-analgesic dose of morphine (30 nmoles), were primarily on the cell membrane (FIG. 6b, upper right panel).

We next assessed whether we could facilitate morphine-induced endocytosis of MOR using DAMGO in vivo. A very low dose of DAMGO was chosen to avoid confusion due to DAMGO-induced endocytosis. Consistent with previous reports (Trafton J. A. et al. (2000) J Neurosci 20:8578-8584), DAMGO, at a dose of 0.01 nmoles, produced neither significant antinociception in the tail-flick assay (FIG. 6a) nor detectable MOR endocytosis in lamina II neurons (FIG. 6b, lower left panel). However, this low dose of DAMGO when administrated concurrently with 30 nmoles of morphine, elicited a remarkable endocytosis of MOR in the spinal cord neurons (FIG. 6b, lower right panel). Quantification of vesicles is listed below each image and was achieved by encoding the slides, and having a second party count vesicles from a center section of a Z stack for at least 8 cells per condition from 2 rats per condition. These results clearly demonstrate that DAMGO and morphine differentially regulate MOR trafficking in the spinal cord and that DAMGO can facilitate morphine-induced endocytosis in vivo thereby altering the RAVE value of morphine.

### Example 8

DAMGO Reduces the Development of Morphine Tolerance in vivo.

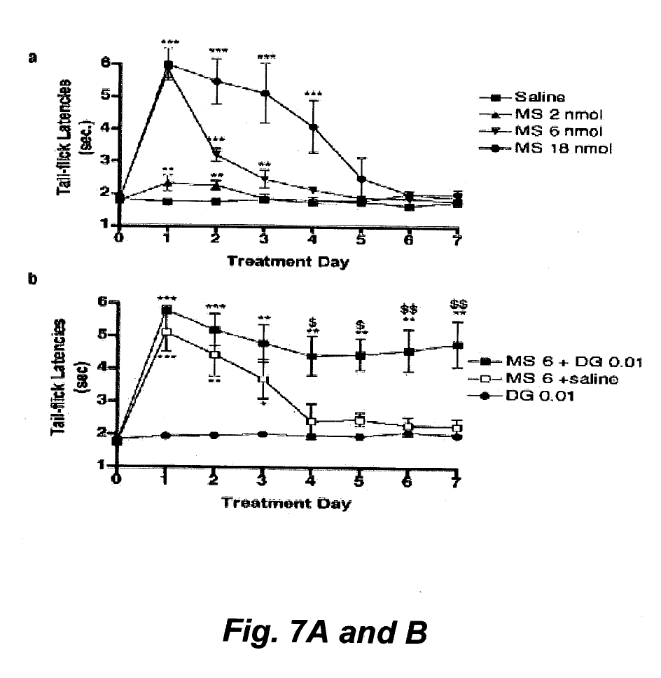

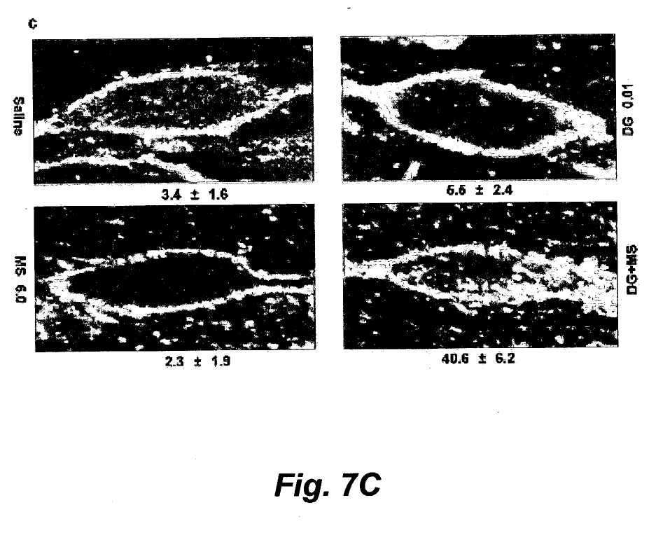

Using these observations, we designed a set of experiments to examine whether alteration of the RAVE value of morphine by DAMGO-mediated dragging would affect the development of morphine tolerance. We first examined the time course of intrathecal morphine tolerance. As in Example 7, rats were implanted with an IT catheter and a time course of morphine tolerance development was assessed with daily tail flick latency testing before pump implantation (day 0) and for 7 consecutive days. Morphine was chronically infused at 2, 6, or 18 nmoles/hr. As shown in FIG. 7A, morphine at all three doses produced a significant antinociceptive effect for the first few days. However, antinociception was gradually reduced during continuous exposure to morphine and was eventually lost completely over 7 days, indicating that the rats had developed tolerance to morphine.

To examine whether the combination of DAMGO and morphine that stimulated endocytosis of MOR, as described in Example 7, could reduce the development of tolerance to chronic morphine administration, we designed the following experiment. Rats were implanted with a Y-shaped intrathecal catheter. One arm of the Y was connected to a mini pump through which either chronic morphine or saline was administered. Either a sub-analgesic, sub-endocytic dose of DAMGO (0.01 nmoles in 15 μl) or an equal volume of saline was administered twice daily through the other arm of the catheter. Twice daily injection of 0.01 nmoles of DAMGO produced no analgesia in the rats receiving saline from their mini pumps (FIG. 7b closed circles), consistent with the inability of this dose of DAMGO to produce antinociception acutely (FIG. 6a). Analgesia was measured by tail flick latency test once per day 30 minutes following the second injection. Rats receiving morphine chronically through their mini pumps and twice daily injections of saline through the catheter showed pronounced antinociception early in the experiment but developed tolerance to the effects of morphine within 4 days (FIG. 7b, open squares). Rats receiving the same dose of morphine through their minipumps and also twice daily injection of 0.01 nmoles of DAMGO through their catheters showed antinociception on day one comparable to that in the rats receiving saline injections. However, remarkably, these rats did not develop tolerance to morphine during the seven days of this experiment (FIG. 7b closed squares).

We hypothesized that the failure of the rats receiving both morphine and DAMGO to develop tolerance was a reflection of the ability of a low dose of DAMGO to alter the RAVE value of morphine by stimulating receptor endocytosis. To examine this possibility, we examined the distribution of the MORs in the spinal cord of the rats from the behavioral experiment. The distribution of MORs in spinal cord neurons of the rats receiving twice daily injection of 0.01 nmoles DAMGO and saline in the mini pump was indistinguishable from animals given only saline (FIG. 7c compare top two panels) and were predominantly on the cell surface, consistent with the results obtained with this dose of DAMGO acutely. The MORs in the spinal cord neurons of the rats given chronic morphine were also predominantly on the cell surface (FIG. 7c lower left panel) consistent with the results obtained with acute morphine in spinal cord neurons (see FIG. 6B, upper right panel). In contrast, the MORs in the spinal cord neurons of the rats with a morphine mini pump, and that received twice daily injection of 0.01 nmoles DAMGO, were distributed not only on the plasma membrane but also within intracellular compartments, suggesting that MORs in these rats were undergoing endocytosis in response to a low dose of DAMGO in combination with chronic morphine. Taken together these results imply that a sub-analgesic dose of a MOR agonist that promotes receptor endocytosis can facilitate the endocytosis of morphine-activated receptors in the cell, thereby decreasing the RAVE value of morphine and reducing the development of tolerance.

We have now shown that a small, sub-analgesic dose of DAMGO, an agonist that promotes endocytosis of the MOR, facilitates morphine-induced endocytosis in trans and thereby lowers the RAVE value of morphine and reduces the development of tolerance. We propose that oligomerization of the MOR influences the endocytic properties of the receptor and, as a consequence of this altered endocytosis, the development of tolerance to morphine is reduced. Although we can not rule out the possibility that other mechanisms associated with the interaction of DAMGO and morphine could be affecting the development of tolerance to morphine, we favor the hypothesis that the rats treated with both drugs become less tolerant to the analgesic effects of morphine as a consequence of the decreased RAVE value of morphine. These results are consistent with our previous studies in cell culture that have demonstrated that increases in endocytosis reduce tolerance and withdrawal. However, these data provide the first in vivo evidence that suggests that alterations in the trafficking properties of the MOR in response to morphine can affect the development of tolerance in an animal model of behavior.

It is likely that tolerance to opiate drugs, as well as other compounds that target GPCRs is mediated by a complex set of mechanisms. We have previously shown that tolerance to morphine in a cell culture model can occur by at least two distinct mechanisms depending on the endocytic and post-endocytic properties of the receptor in response to morphine (Finn et al. (2001) Neuron 32:829-839). Furthermore, Yobum and colleagues have demonstrated that opiate tolerance can occur by receptor density-dependent and—independent mechanisms depending on whether or not the agonist used promotes endocytosis (Stafford et al. (2001) Pharmacol Biochem Behav 69:233-237).

Tolerance to morphine can occur as a result of superactivation of the adenylyl cyclase signaling pathway (Sharma et al. (1975) Proc Natl Acad Sci USA 72:3092-3096), which masks morphine's effect by altering the homeostatic baseline of the MOR expressing cells. Several groups have reported superactivation of the cAMP signaling pathway in response to chronic morphine treatment in brain regions implicated in addiction, including the locus coeruleus (Nestler (1996) Neuron 16:897-900), ventral tegmental area (Bonci et al. (1997) J Neurosci 17:796-803), nucleus accumbens (Chieng et al. (1998) J Neurosci 18:7033-7039; Terwilliger et al. (1991) Brain Res 548:100-110), amygdala (Terwilliger et al. (1991) Brain Res 548:100-110) and dorsal raphe (Jolas et al. (2000) Neuroscience 95:433-443). Cellular changes occurring during cAMP superactivation include increased expression of certain adenylyl cyclases, PKA, and CREB (reviewed in (Nestler (2001) Nat Rev Neurosci 2:119-128; Williams et al. (2001) Physiol Rev 81:299-343)). These adaptive cellular changes compensate for continued inhibition of adenylyl cyclase, and are functionally analogous since they serve to increase the amount of signaling through the cAMP pathway and thus subvert the effect of morphine. This cellular tolerance is clearly revealed upon removal of drug whereby the superactivation manifests itself as withdrawal. Superactivation following drug withdrawal demonstrates that the MORs in these cells are still coupled to second messenger cascades when drug is present and hence this form of tolerance would be receptor density independent. This cellular tolerance is alleviated by receptor endocytosis ((Finn et al. (2001) Neuron 32:829-839).

On the other hand, tolerance to morphine could also occur as a result of receptor desensitization or receptor downregulation. Tolerance mediated solely by receptor desensitization would lead to reduced receptor-mediated signaling without a loss of surface receptors. Several groups have reported reduced MOR-mediated signaling in various brain regions following chronic morphine treatment often without a concomitant loss in receptor number (Christie et al. (1987) Mol Pharmacol 32:633-638; Connor et al. (1999) Br J Pharmacol 126:1553-1558; Selley et al. (1997) Brain Res 746:10-18; Sim et al. (1996) J Neurosci 16:2684-2692). Tolerance mediated by receptor downregulation would lead to reduced receptor-mediated signaling because of a loss of surface receptors. Several groups have reported that in some brain regions there is, in fact, a loss of receptors following prolonged morphine treatment (Abdelhamid et al. (1991), Eur J Pharmacol 198:157-163; Bernstein et al. (1998) Brain Res Mol Brain Res 55:237-242; Tao et al. (1998) Eur J Pharmacol 344:137-142). However in other regions receptor number remains unchanged (De Vries et al. (1993) Life Sci 52:1685-1693; Simantov et al. (1984) Neuropeptides 5:197-200; Werling et al. (1989) Proc Natl Acad Sci USA 86:6393-7) or is even upregulated in tolerant animals (Brady et al. (1989) Brain Res 477:382-386; Gouarderes et al. (1990) Prog Clin Biol Res 328:175-178; Rothman et al. (1991) Peptides 12:151-160; Tejwani et al. 1998) Brain Res 797:305-312). It is likely that all these mechanisms, and potentially others as well, contribute to opiate tolerance. Furthermore, although cellular mechanisms including receptor number, desensitization and homeostasis can contribute to tolerance, additional complex mechanisms involving alterations in neuronal circuitry are likely involved in the development of associative tolerance (Mitchell et al. (2000) Nat Neurosci 3:47-53).

The observation that beta-arrestin 2 knock-out mice show reduced analgesic tolerance (Bohn et al. (2000) Nature 408:720-723) suggests that, in certain cell types, receptor desensitization may contribute directly to morphine tolerance perhaps by serving as a first step towards receptor downregulation, although receptor number was not assessed in these animals. These data are consistent with the prevailing hypothesis that receptor desensitization contributes directly to tolerance. However, it is important to note that the endocytic trafficking of several classes of GPCR are likely also affected by the loss of beta-arrestin in these animals many of which may also be involved in pain transmission. Furthermore, the beta-arrestin 2 knock-out mice still demonstrate withdrawal from morphine, as assessed biochemically with cAMP superactivation. Hence, cellular tolerance is still occurring in these animals even though behavioral tolerance is reduced. Clearly, the emerging picture of regional differences in the extent of chronic morphine-induced MOR desensitization (Sim et al. (1996) J Neurosci 16:2684-2692; Sim-Selley et al. (2000) J Neurosci 20:4555-4562) as well as regionally distributed splice variants differing in their cytoplasmic tails (Abbadie et al. (2000) J Comp Neurol 419:244-256) promises to impart considerable complexity to the biochemical characterization of the processes of desensitization and superactivation in different brain regions.

To our knowledge, this is the first study to demonstrate that increased MOR endocytosis in response to morphine can reduce the development of tolerance in an animal model. These results have important implications for the treatment of chronic pain. First they suggest that agonists that promote endocytosis of the MOR might provide analgesics with reduced liability for tolerance. This is in contrast to the prevailing hypothesis that desensitization and endocytosis of the MOR contributes directly to tolerance by decreasing the number of functional receptors. It is important to note that agonists that promote desensitization of receptors are routinely discarded in drug discovery programs precisely because of this prevailing view. However, even without the development of new opiate analgesics, the results here suggest that the development of tolerance to morphine can be delayed by the co-administration of drugs that promote endocytosis. In short, our results suggest that two drugs actually produce less tolerance than morphine alone.

### Example 9

Morphine Tolerance and Wthdrawal in the Intracerebroventricular (i.c.v.) Cannula Model.

Pain transmission is mediated at two primary sites: the spinal cord and the brain. Studies using intrathecal catheters allow assessment of the effects of drugs and/or agonists delivered directly to the spinal cord. The effects of drugs and/or agonists delivered to the brain can be assessed by intracerebroventricular (i.c.v.) cannula implantation. The i.c.v. model provides supplemental information on morphine-induced analgesia and related tolerance. Furthermore, analgesia tolerance is only one of several opioid addiction-associated problems, which also include physical dependence and symptoms of opiate withdrawal. Withdrawal is primarily mediated at the brain level and hence, the i.c.v. model permits the investigation of dependence/withdrawal, as well as tolerance.

Intracerebroventricular (i.c.v.) cannula implantation. Rats were anesthetized with halothane and placed in a stereotaxic head holder. The scalp was shaved and scrubbed with alcohol. A midline sagittal incision was made, and the skull was exposed. The bone suture junctions Bregma and midline (lambda) were identified, then the location for cannula placement was marked 1 mm posterior to Bregma and 1.5 mm lateral (left) of the midline. A hole was drilled through the skull at the marked location to receive the cannula. Then, three additional holes were drilled around the first hole: anterior, lateral and posterior to the first hole, respectively, and about 3-4 mm apart from the first hole. These three holes were drilled partway through the skull to accommodate three small screws used to secure the cannula. An L-shaped cannula was inserted through the first hole in the skull to the stereotaxically correct depth 3.6 mm below the surface of the skull. The skull surface was completely dried, and the cannula and the implantation site covered with dental cement. Once the surgical procedures were done, the rat was removed from the stereotaxic device and returned to its cage and allowed 5 to 7 days to recover from surgery.

Implantation of osmotic mini-pump. After 5 to 7 days of recovery, a subcutaneous pocket in the midscapular area of the back of each rat, next to the implanted cannula (caudally), was created under light anesthesia with halothane to house the osmotic mini-pump. The subcutaneous pocket was created by first making a small incision, inserting a hemostat into the incision, and then opening and closing the hemostat to make a short subcutaneous tunnel. Finally, a mini-pump pre-filled with either saline or morphine was implanted into the pocket and the mini-pump connected to the cannula. The wound was closed with sutures and the rat was returned to its cage. After implantation, morphine or saline is released continuously from the mini-pump into the brain.

A. Morphine Given i.c.v. Causes Tolerance.

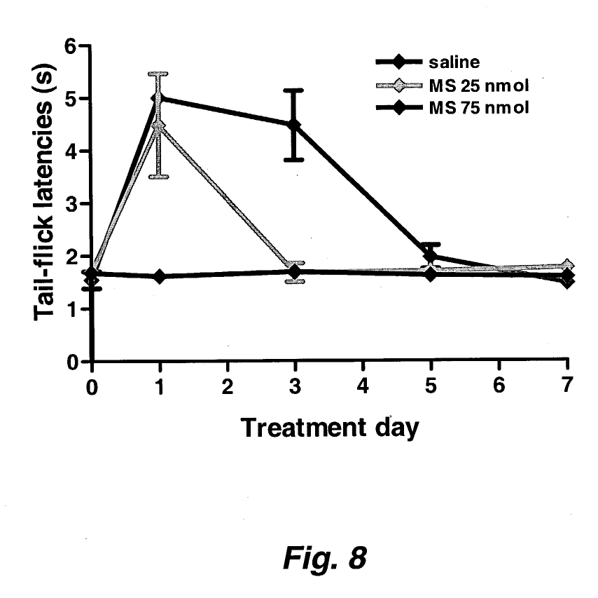

After mini-pump implantation, morphine or saline was infused chronically for 7 consecutive days. Morphine was infused at 25 or 75 nmoles/hr. The time course of morphine tolerance was assessed with daily tail flick latency testing starting before mini-pump implantation (day 0). As shown in FIG. 8, morphine produced a significant antinociceptive effect for the first 3-4 days. However, continuous exposure to morphine resulted in the loss of antinociception, with the loss occurring sooner at the lower dose (25 nmoles/hr). Thus, morphine given i.c.v. causes tolerance in a dose-dependent manner.

B. Morphine Given i.c.v. Produces Withdrawal.

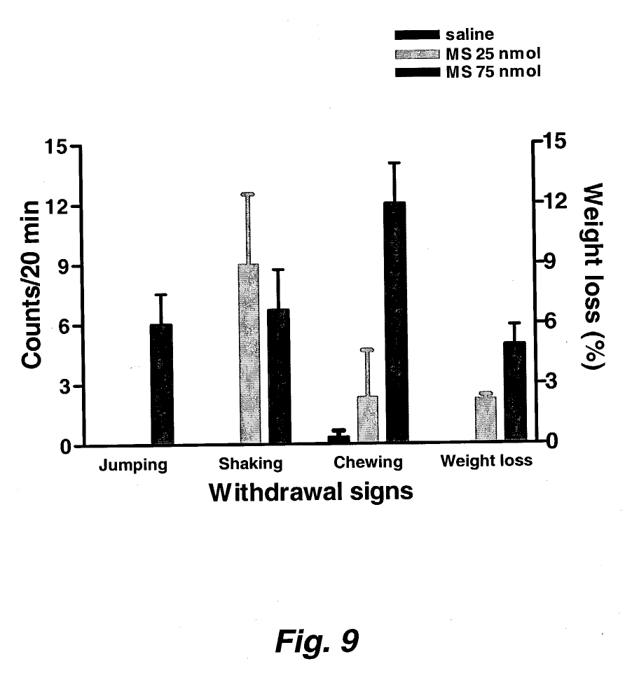

After mini-pump implantation, morphine or saline was infused chronically for 7 consecutive days. Morphine was infused at 25 or 75 nmoles/hr. On day 7, rats were injected intraperitoneally with 3 mg/kg naloxone and placed, individually, in Plexiglass cylinders. The rats were monitored for jumping, shaking, and chewing, and the number of occurrences of each type of behavior over a 20-min period was recorded immediately following the naloxone injection. In addition, the rats were weighed before naloxone injection and after the 20 mins of monitoring indicated above. FIG. 9 shows that treatment with naloxone, which blocks morphine's effects produced increases in jumping, shaking, and chewing in rats receiving the higher dose of morphine (75 nmoles/hr) and increases in shaking and chewing in rats receiving the lower dose (25 nmoles/hr). In addition, naloxone treatment produced dose-dependent weight loss in morphine-treated rats. As these three behaviors and weight loss are symptoms of withdrawal (see, e.g., Nitsche J F, et al., J Neurosci 22(24):10906-13 (Dec. 15, 2002) “Genetic dissociation of opiate tolerance and physical dependence in delta-opioid receptor-1 and preproenkephalin knock-out mice.”), these data indicate that the 7-day i.c.v. morphine treatment produced physical dependence in a dose-dependent manner.

C. DAMGO Produces Less Tolerance than Morphine.

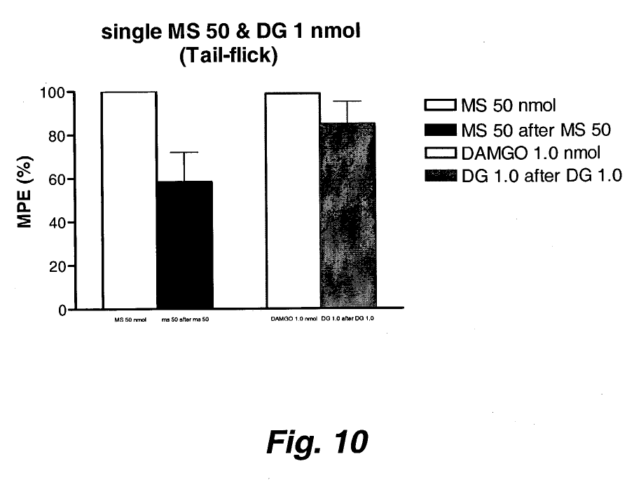

To compare the tolerance produced by DAMGO, which promotes MOR endocytosis, with that produced by morphine, which does not, rats were implanted with i.c.v. cannulae as described above (but not with mini-pumps). Morphine (MS, 50 nmoles) or DAMGO (DG, 1 nmole) was given directly via cannula, twice a day: morning and afternoon, in 5 μl volume for 5 days. Rats were tested for analgesia using the tail-flick latency test twice during the study period. The tests were conducted 30 min after the morning dose on days 1 and 5. Maximum Possible Effect (MPE) was calculated using the following equation: (Post-drug latency—baseline latency)/(cut-off latency—baseline latency)×100%. The results are shown in FIG. 10. The percent MPE is shown for tail flick latency tests conducted after the initial morphine (MS 50 nmol) and DAMGO (DAMGO 1.0 nmol) treatments and after 5 days of treatment with morphine (ms 50 after ms 50) and DAMGO (DG 1.0 after DG 1.0). 5-day morphine treatment results in a reduction in antinociceptive effect by about 30-40%, whereas 5-day DAMGO treatment results in a much smaller reduction in antinociception. These data indicate that DAMGO produces less tolerance than morphine.

D. DAMGO Produces Less Withdrawal than Morphine.

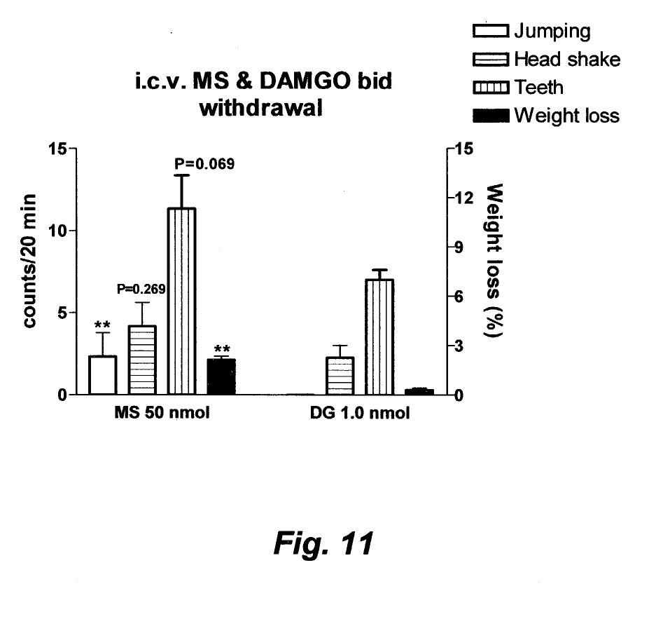

To compare any withdrawal produced by DAMGO, which promotes MOR endocytosis, with that produced by morphine, which does not, rats were treated i.c.v. with morphine (MS, 50 nmoles) and DAMGO (1.0 nmole) twice daily for 5 days as described in Example 9.C. On day 5, 30 mins following the second drug administration, rats were injected intraperitoneally with 3 mg/kg naloxone and placed, individually, in Plexiglass cylinders. The rats were monitored for jumping, shaking, and chewing, and the number of occurrences of each type of behavior over a 20-min period was recorded immediately following the naloxone injection. In addition, the rats were weighed before naloxone injection and following the 20-min observation period indicated above. FIG. 11 shows that treatment with naloxone, which blocks morphine's effects, produced jumping, shaking, and chewing and weight loss in rats receiving morphine. In contrast rats receiving DAMGO exhibited no jumping and less shaking, chewing, and weight loss that morphine-treated rats. Thus, by four indicators of withdrawal, DAMGO produces less withdrawal, indicating less physical dependence, than morphine.

E. Morphine-Induced Tolerance is not Associated with a Decrease in MOR Number in the Brain.

Rats were treated chronically i.c.v. with morphine or saline for 7 consecutive days as described in Examples 9.C. and 9.D. Morphine was administered by mini-pump at 25 or 75 nmoles/hr for 7 consecutive days, as described in Examples 9.A. and 9.B. to induce tolerance. After the behavioral study described in Example 9.B., the rats were sacrificed, and the brains were quickly removed and frozen by immersion in isopentane on dry ice and stored at −80° C. Brain sections, 16 μm thick, were cut on a cryostat at −18° C., thaw-mounted onto slides, and stored desiccated at −80° C.

A MOR receptor binding assay was carried out using slides containing sections from the midbrain, forebrain, and brain stem as follows:

1. Slides containing brain sections were pre-incubated in buffer (50 mM Tris-HCl, pH 7.4) for 30 min at 25° C.

2. The slides were incubated in the same buffer containing 5 nM of [³H] DAMGO for 60 min at room temperature.

3. Non-specific binding was assessed on adjacent sections treated with 1 μM naloxone to block MOR binding.

4. The slides were then rinsed for 2-5 min in ice-cold buffer (50 mM Tris-HCl, pH 7.4) and dipped in cold distilled water.

5. The slides were thoroughly dried overnight, placed in X-ray cassettes, and exposed to BioMax MS™ film with an intensifying screen for 3 weeks.

6. After exposure, the films were developed and scanned. The regional neuroanatomy of the rat was determined with the atlas of Paxinos and Watson, and brain areas in autoradiograms were quantitated using NIH Image software and optical densities were converted into fmol/mg tissue according to commercial standards exposed adjacent to the brain sections.

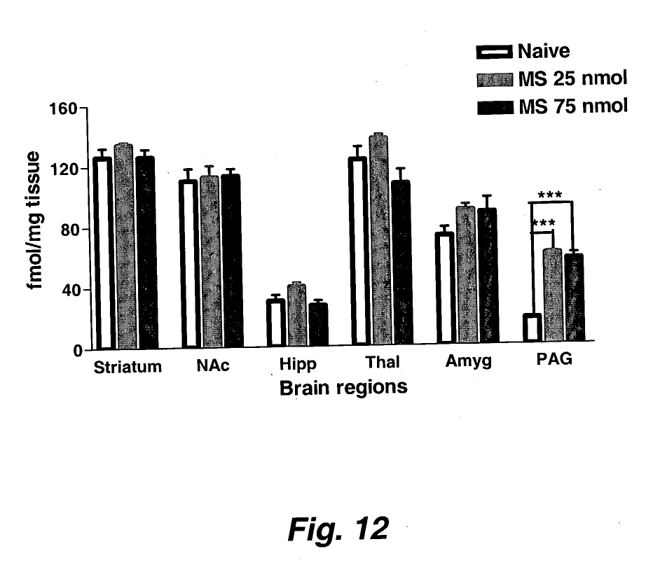

FIG. 12 is a histogram showing the results of this study for different brain regions: the striatum, the nucleus accumbens (NAc), the hippocampus, the thalamus, the amygdala, and the brain stem (PAG). Results are shown for rats treated with saline (naïve) or 25 or 75 nmoles/hr morphine for 7 days (MS 25 nmol and MS 75 nmol, respectively). Chronic morphine treatment sufficient to induce tolerance does not result in a reduction in receptor number. In fact, chronic morphine treatment was correlated with a significant increase in receptor number in the brain stem (PAG). Thus, morphine-induced tolerance does not appear to be associated with a decrease in receptor number.

F. Morphine-Induced Tolerance and MOR-G Protein Coupling in the Brain.

To examine whether morphine-induced tolerance is associated with changes in MOR-G protein coupling, brain sections from rats treated chronically (twice daily) with morphine or DAMGO (see Example 9.C. for treatment details) were prepared as described in Example 9.E. and assayed for binding with [³⁵S ]-GTPγS binding. The binding assay was carried out as follows:

1. Slides containing brain sections were pre-incubated in buffer (50 mM Tris, 3 mM MgCl₂, 0.2 mM EGTA, 100 mM NaCl, pH 7.4) for 10 min at room temperature.

2. The slides were then pre-incubated in the same buffer containing 2 mM GDP for 10 min at room temperature.

3. The slides were incubated in the same buffer as in step 2, additionally containing 50 pM [³⁵S]GTPγS for 90 min at room temperature.

4. The slides were then rinsed twice for 2-5 min in ice-cold buffer (the same buffer as in step 1) and dipped in cold distilled water.

5. The slides were thoroughly dried overnight, placed in X-ray cassettes, and exposed to BioMax MS™ film with an intensifying screen for 72 hr.

6. After exposure, the films were developed and scanned. The regional neuroanatomy of the rat was determined with the atlas of Paxinos and Watson, and brain areas in autoradiograms were quantitated using NIH Image software. Percent stimulation was calculated from optical densities (OD) according to the following equation:

Percent stimulation=(stimulated OD−basal OD)/basal OD×100%.

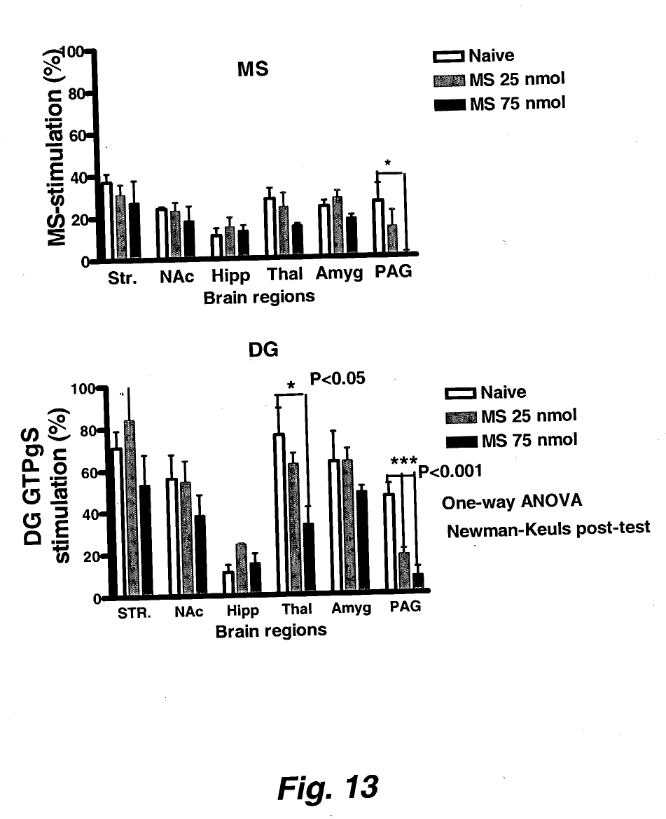

FIG. 13 is a histogram showing the results of this study for different brain regions: the striatum, the nucleus accumbens (NAc), the hippocampus, the thalamus, the amygdala, and the brain stem (PAG). The top panel (13.A.) shows morphine-stimulation of GTPγS binding, and the bottom panel (13.B.) shows DAMGO stimulation of GTPγS binding. Results are shown for rats treated for 7 days with saline (naïve) or 25 or 75 nmoles/hr morphine (MS 25 nmol and MS 75 nmol, respectively). Chronic morphine treatment sufficient to induce tolerance does not result in MOR-G protein uncoupling in the midbrain. There is a significant (P<0.05) reduction in MOR-G protein coupling in the brain stem (PAG), where Example 9.E. showed an increase in receptor number, suggesting that, while more receptors are present, fewer couple with G protein. Chronic DAMGO treatment is associated with a reduction MOR-G protein coupling in the brainstem (P<0.001) and in the thalamus (P<0.05).

G. MOR Distribution Following Acute and Chronic Treatment with Morphine and DAMGO.

To examine MOR distribution after acute and chronic treatment with morphine as compared to DAMGO, rats were implanted with i.c.v. cannulae. Morphine (MS, 50 nmoles) or DAMGO (DG, 1 nmole) was given directly via cannula. For the acute treatment, animals were sacrificed 30 mins following the first injection. For the chronic treatment, drugs were given twice a day: morning and afternoon, in 5 μl volume for 5 days, and the animals were sacrificed 30 mins following the final injection. Rats were deeply anesthetized with halothane and perfused with 4% paraformaldehyde in 0.1 M phosphate buffer. The brains were dissected out and post-fixed overnight in the same fixative and then transferred to a 30% sucrose buffer. Coronal sections (30 um thick) were cut on cryostat at −18° C., preincubated in PBT solution (0.1 M phosphate buffer, 2% BSA, and 0.2% Triton X-100) for 30 min, blocked in 5% normal goat serum in PBT solution for another 30 min, and then incubated with a rabbit anti-mu opioid receptor antibody at 1:5000 and mouse and NeuN antibody (which recognizes the neuronal-specific protein NeuN) at 1:5000 overnight at 4° C. The sections were washed several times with PBT and incubated in Alexa Fluor 488 goat anti rabbit antibody for mu-opioid receptor (green) and Alexa Fluor 546 goat anti mouse antibody for NeuN (red) for 2 hours at room temperature. The sections were then washed and mounted onto slides. The mu-opioid receptors and NeuN were visualized using a Zeiss confocal microscope with a 60× oil immersion objective.

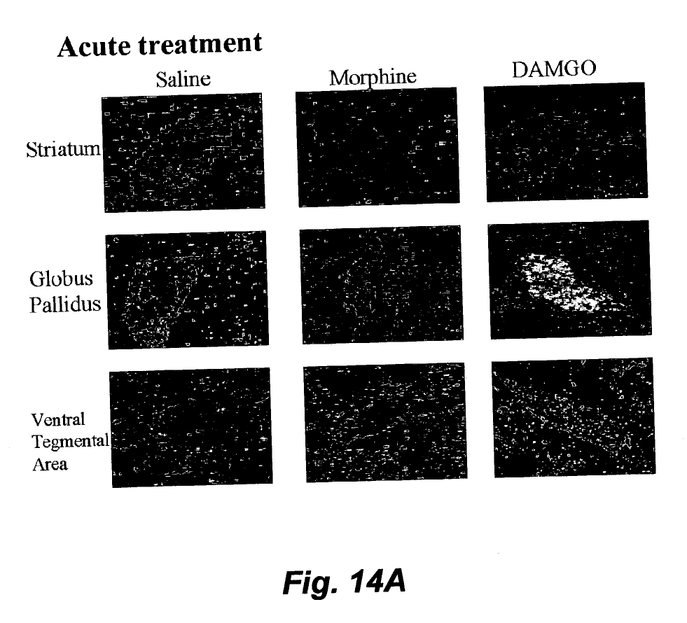

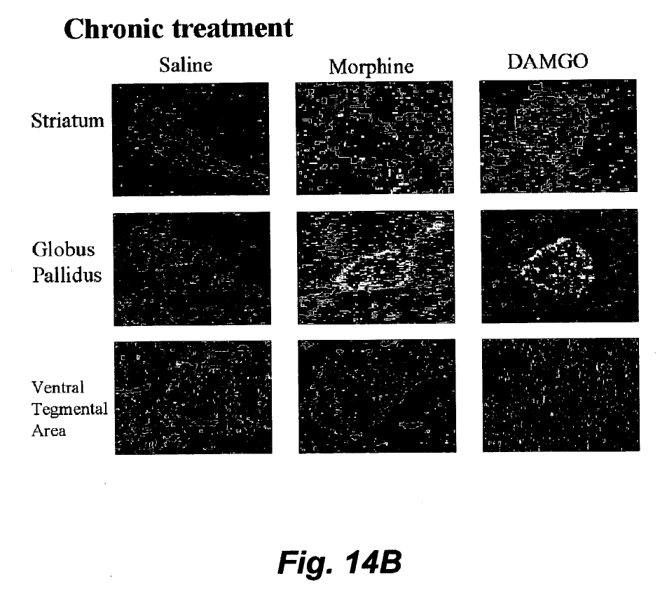

FIG. 14.A. shows MOR distribution (green) for three brain regions, the striatum, the globus pallidus, and the ventral tegumental area, after acute treatement with saline, morphine, or DAMGO. NeuN distribution (red) indicates the location of neurons. FIG. 14.B. shows MOR (green) and NeuN distribution (red) for the same regions after chronic treatment with saline, morphine, or DAMGO. MOR endocytosis is indicated by an increase in the green signal within the cell boundaries (which are stained more intensely green). These results demonstrate that morphine, administered acutely or chronically, does not promote substantial MOR endocytosis, whereas DAMGO does.

Although the invention has been described herein with reference to particular embodiments, it is to be understood that these embodiments are merely illustrative of the various aspects of the invention. It will be understood that numerous modifications may be made of the illustrative embodiments and other arrangements may be devised without departing from the spirit and the scope of the invention.

All publications and patents mentioned in this specification are herein incorporated by reference to the same extent as if each individual publication or patent were specifically and individually indicated to be incorporated by reference.

What is claimed is:

1. A pharmaceutical composition comprising:
a drug that targets a G protein-coupled receptor (GPCR), wherein the drug does not promote endocytosis and resensitization of the targetted GPCR;
an agonist for the GPCR, wherein the agonist promotes the endocytosis of the GPCR and is present in the composition in an amount sufficient to promote endocytosis and resensitization of the targetted GPCR; and
a pharmaceutically acceptable carrier.

2. The pharmaceutical composition of claim 1, wherein the drug activates the GPCR.

3. The pharmaceutical composition of claim 1, wherein the drug comprises an analgesic and is present in an analgesic amount.

4. The pharmaceutical composition of claim 1, wherein the agonist comprises an analgesic and is present in a sub-analgesic amount.

5. The pharmaceutical composition of claim 1, wherein the drug comprises an opioid drug, the GPCR comprises the mu opioid receptor, and the agonist comprises a mu opioid receptor agonist.

6. The pharmaceutical composition of claim 5, wherein the opioid drug activates the mu opioid receptor.

7. The pharmaceutical composition of claim 5, wherein the opioid drug comprises an analgesic and is present in an analgesic amount.

8. The pharmaceutical composition of claim 7, wherein the mu opioid receptor agonist comprises an analgesic and is present in a sub-analgesic amount.

9. The pharmaceutical composition of claim 5, wherein the opioid drug comprises morphine.

10. The pharmaceutical composition of claim 9, wherein the agonist comprises a compound selected from the group consisting of DAMGO, methadone, fentanyl, sufentanil, remi-fentanyl, etonitazene, and etorphine.

11. A method for reducing, preventing or delaying the development of tolerance to, and/or physical dependence on, a drug that targets a G protein-coupled receptor (GPCR), wherein said tolerance and/or physical dependence develops as a consequence of the failure of the drug to promote endocytosis and resensitization of the targetted GPCR, said method comprising achieving co-administration of the drug and an agonist for the GPCR in a subject by:
administering the drug to a subject receiving the agonist;
administering the agonist to a subject receiving the drug; or
administering the drug and the agonist to a subject;
wherein the agonist promotes the endocytosis of the GPCR and is present in the subject in an amount sufficient to promote endocytosis of the GPCR, whereby the drug-targetted GPCR is endocytosed and resensitized;

12. The method of claim 11, wherein the drug activates the GPCR.

13. The method of claim 11, wherein the drug comprises an analgesic and is present in the subject in an analgesic amount.

14. The method of claim 13, wherein the agonist comprises an analgesic and is present in the subject in a sub-analgesic amount.

15. The method of claim 11, wherein the drug comprises an opioid drug, the GPCR comprises the mu opioid receptor, and the agonist comprises a mu opioid receptor agonist.

16. The method of claim 15, wherein the opioid drug activates the mu opioid receptor.

17. The method of claim 15, wherein the opioid drug comprises an analgesic and is present in the subject in an analgesic amount.

18. The method of claim 17, wherein the mu opioid receptor agonist comprises an analgesic and is present in the subject in a sub-analgesic amount.

19. The method of claim 15, wherein the opioid drug comprises morphine.

20. The method of claim 19, wherein the agonist comprises a compound selected from the group consisting of DAMGO, methadone, fentanyl, sufentanil, remifentanyl, etonitazene, and etorphine.

21. A method of screening for an agent that reduces, prevents or delays the development of tolerance to, and/or physical dependence on, a drug that targets a G protein-coupled receptor (GPCR), said method comprising:
contacting a test agent with a cell comprising the GPCR;
determining whether the test agent promotes the endocytosis of the GPCR;
selecting a test agent that promotes endocytosis of the GPCR as an agent that may reduce, prevent or delay the development of tolerance to, and/or physical dependence on, the drug.

22. The screening method of claim 21, wherein said method additionally comprises recording any selected test agent in a database of agents that may reduce, prevent or delay the development of tolerance to, and/or physical dependence on, the drug.

23. The screening method of claim 21, wherein the test agent comprises an analgesic.

24. The screening method of claim 21, wherein the GPCR comprises the mu opioid receptor, and the test agent comprises a mu opioid receptor agonist.

25. The screening method of claim 21, wherein said contacting is in vitro.

26. The screening method of claim 21, wherein said determining comprises determining receptor endocytosis by a ligand binding assay.

27. The screening method of claim 21, additionally comprising combining a selected test agent with a pharmaceutically acceptably carrier.

28. The screening method of claim 21, additionally comprising combining a selected test agent with a drug that targets the GPCR, wherein the drug does not promote endocytosis of the GPCR.
